(19) United States
(12) Patent Application Publication
(10) Pub. No.: US 2025/0172991 A1
(43) Pub. Date: May 29, 2025

(54) INFORMATION PROCESSING APPARATUS, INFORMATION PROCESSING METHOD, AND PROGRAM

(71) Applicant: SONY GROUP CORPORATION, TOKYO (JP)

(72) Inventor: KIYOSHI YOSHIKAWA, TOKYO (JP)

(21) Appl. No.: 18/841,656

(22) PCT Filed: Feb. 15, 2023
(86) PCT No.: PCT/JP2023/005120

§ 371 Date: Aug. 26, 2024

(30) Foreign Application Priority Data
Mar. 1, 2022 (JP) 2022-030583

(51) Int. Cl. G06F 3/01; B25J 11/00; G06F 3/16

(52) U.S. Cl. CPC G06F 3/011; B25J 11/0005; G06F 3/165

(57) ABSTRACT

The present technique relates to an information processing apparatus, an information processing method, and a program capable of predicting a future state of a user and supporting the user. A supporting unit is configured to support a user based on an estimation result of at least one of a present affect and a present context of the user and a prediction result of at least one of a future affect and a future context of the user. For example, the present technique can be applied to an audio agent apparatus of an audio support system which supports the user by transmitting by audio, in response to a request from the user by an utterance or the like, a message that is most readily receivable by the user as a response.

### TECHNICAL FIELD

The present technique relates to an information processing apparatus, an information processing method, and a program, and particularly to an information processing apparatus, an information processing method, and a program capable of predicting a future state of a user and supporting the user.

### BACKGROUND ART

In user support agents including AI (Artificial Intelligence) assistants, a present state of a user must be accurately estimated or a future state of the user must be predicted in order to appropriately support the user. However, it is difficult to accurately estimate a state of the user solely based on a facial expression of the user or contents of an utterance by the user.

In consideration thereof, there is an information processing system which recognizes a present context of a user based on at least any of a surrounding environment of the user, an affect of the user, a state of the user, and an affect or a state of a person near the user (for example, refer to PTL 1).

### CITATION LIST

### Patent Literature

### PTL 1

WO 2018/021040

### SUMMARY

### Technical Problem

However, it is difficult to predict a future state of a user in such an information processing system. As a result, while there is a need to provide a method capable of predicting a future state of a user and supporting the user, the status quo is that such a need is not sufficiently satisfied.

The present technique has been devised in such circumstances and enables a future state of a user to be predicted and the user to be supported.

### Solution to Problem

An information processing apparatus or a program according to an aspect of the present technique is an information processing apparatus including a supporting unit configured to support a user based on an estimation result of at least one of a present affect and a present context of the user and a prediction result of at least one of a future affect and a future context of the user or a program causing a computer to function as the information processing apparatus.

An information processing method according to an aspect of the present technique is an information processing method including an information processing apparatus performing a supporting step of supporting a user based on an estimation result of at least one of a present affect and a present context of the user and a prediction result of at least one of a future affect and a future context of the user.

According to an aspect of the present technique, a user is supported based on an estimation result of at least one of a present affect and a present context of the user and a prediction result of at least one of a future affect and a future context of the user.

### BRIEF DESCRIPTION OF DRAWINGS

FIG. 1 is a block diagram illustrating a configuration example of an audio support system to which the present technique is applied.

FIG. 2 is a diagram illustrating Russell's circumplex model of affect.

FIG. 3 is a diagram illustrating an example of information stored in a database illustrated in FIG. 1.

FIG. 4 is a diagram illustrating an example of an outline of processing by the audio support system.

FIG. 5 is a flowchart describing audio support processing.

FIG. 6 is a block diagram illustrating a configuration example of a bicycle navigation system to which the present technique is applied.

FIG. 7 is a diagram illustrating an example of information stored in a database illustrated in FIG. 6.

FIG. 8 is a diagram illustrating an example of an outline of processing by the bicycle navigation system.

FIG. 9 is a block diagram illustrating a configuration example of a pet-type robot system to which the present technique is applied.

FIG. 10 is a diagram illustrating an example of information stored in a database illustrated in FIG. 9.

FIG. 11 is a diagram illustrating an example of an outline of processing by the pet-type robot system.

FIG. 12 is a block diagram illustrating a configuration example of a driving support system to which the present technique is applied.

FIG. 13 is a diagram illustrating an example of information stored in a database illustrated in FIG. 12.

FIG. 14 is a diagram illustrating an example of an outline of processing by the driving support system.

FIG. 15 is a block diagram illustrating a configuration example of a cooking support system to which the present technique is applied.

FIG. 16 is a diagram illustrating an example of information stored in a database illustrated in FIG. 15.

FIG. 17 is a diagram illustrating an example of an outline of processing by the cooking support system.

FIG. 18 is a block diagram illustrating a configuration example of hardware of a computer.

### DESCRIPTION OF EMBODIMENTS

Modes for embodying the present technique (hereinafter, referred to as embodiments) will be described below. Note that the description will be presented in the following order.
1. First embodiment (audio support system)
2. Second embodiment (bicycle navigation system)
3. Third embodiment (pet-type robot system)
4. Fourth embodiment (driving support system)
5. Fifth embodiment (cooking support system)
6. Computer

### First Embodiment

### Configuration Example of Audio Support System

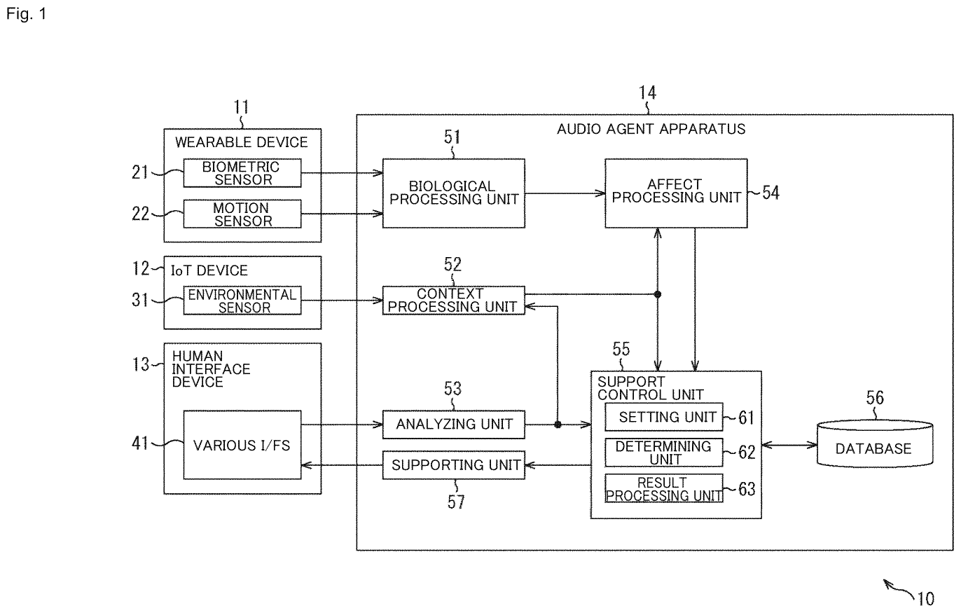

FIG. 1 is a block diagram illustrating a configuration example of an audio support system including an audio agent apparatus as an information processing apparatus to which the present technique is applied.

An audio support system 10 illustrated in FIG. 1 is constituted of one or more wearable devices 11, one or more IoT (Internet of Things) devices 12, one or more human interface devices 13, and an audio agent apparatus 14.

The audio support system 10 supports a user by transmitting by audio, in response to a request from the user by an utterance or the like, a message that is most readily receivable by the user as a response. A message that is most readily receivable by the user is, for example, a message that does not significantly alter a state of the user or, in other words, a message that does not disturb the user.

Specifically, the wearable device 11 of the audio support system 10 is constituted of a smart watch, a hearable device, or the like to be mounted to a part of the body of the user such as a wrist or an ear. The wearable device 11 includes a biometric sensor 21 and a motion sensor 22. The biometric sensor 21 acquires a biosignal that is a signal representing a blood flow, respiration, or the like of the user as detected by methods including EEG (Electroencephalography), ECG (Electrocardiogram), PPG (Photoplethysmogram), EDA (Electro Dermal Activity), and LDF (Laser Doppler flowmetry). The biometric sensor 21 inputs the biosignal to the audio agent apparatus 14.

The motion sensor 22 is constituted of an acceleration sensor, a gyro sensor, or the like. The motion sensor 22 acquires an acceleration or an angular velocity of the biometric sensor 21 as associated biological information that is information accompanying a biosignal. The motion sensor 22 inputs the associated biological information to the audio agent apparatus 14.

Note that the audio support system 10 may include a remote sensing apparatus that performs remote sensing in place of the wearable device 11. In this case, for example, a heart rate or the like of the user is estimated by the remote sensing apparatus that is a Web camera or the like and acquired as a biosignal.

The IoT device 12 includes environmental sensors 31 that are various sensors which acquire environmental information indicating a state of the user or a state of an environment surrounding the user. Examples of the environmental sensors 31 include a GPS (Global Positioning System) sensor, an image sensor, an ultrasonic sensor, an infrared camera, an acceleration sensor, a gyroscope sensor, a temperature/humidity sensor, and a weather sensor. The GPS sensor acquires, for example, information on a present position of the user as environmental information. The image sensor, the ultrasonic sensor, the infrared camera, the acceleration sensor, and the gyroscope sensor are used to acquire, for example, information representing a posture or a motion of the user as environmental information. The temperature/humidity sensor acquires information representing a temperature and a humidity of the surroundings of the user as environmental information. The weather sensor acquires information on the weather around the user as environmental information. The IoT device 12 inputs the environmental information acquired by the environmental sensors 31 to the audio agent apparatus 14.

The human interface device 13 includes an input device that accepts an input from the user and an output device which provides the user with output. Examples of the input device include a microphone, a touch sensor, a pressure sensor, and a keyboard and examples of the output device include a speaker.

The human interface device 13 includes various I/Fs 41 that interact with the user. For example, the various I/Fs 41 of the microphone input information representing an utterance input from the user as input information that is information input from the user to the audio agent apparatus 14. The various I/Fs 41 of the touch sensor inputs information representing contents selected by a touch input by the user as input information to the audio agent apparatus 14. The various I/Fs 41 of the pressure sensor inputs information representing contents selected by a pressing force exerted by the user as input information to the audio agent apparatus 14. The various I/Fs 41 of the keyboard inputs information representing characters input from the user as input information to the audio agent apparatus 14. The various I/Fs 41 of the speaker supports the user by outputting audio to the user based on control information input from the audio agent apparatus 14.

Information exchange between the wearable device 11, the IoT device 12, and the human interface device 13, and the audio agent apparatus 14, is performed via a wired or wireless network.

The audio agent apparatus 14 is constituted of a biological processing unit 51, a context processing unit 52, an analyzing unit 53, an affect processing unit 54, a support control unit 55, a database 56, and a supporting unit 57.

Based on associated biological information input from the motion sensor 22, the biological processing unit 51 of the audio agent apparatus 14 performs a noise determination of the biosignal input from the biometric sensor 21 and removes noise in the biosignal. The biological processing unit 51 extracts various feature amounts to be used to estimate or predict an affect of the user from the biosignal from which noise has been removed. For example, the feature amount is a low-frequency wave (LF), a high frequency wave (HF), or the like when the biosignal is a signal representing a heart rate of the user and an a wave, a θ wave, or the like when the biosignal is a signal representing a brain wave of the user. The biological processing unit 51 supplies the feature amount of the biosignal to the support control unit 55.

The context processing unit 52 estimates a present context of the user due to behavior recognition based on at least one of the environmental information input from the environmental sensor 31 and an analysis result of input information supplied from the analyzing unit 53 and obtains an estimation result.

The context that is estimated at this point ranges from a primitive context directly obtained from the environmental sensor 31 or a single analysis result of input information to context estimated based on a combination of environmental information and an analysis result of input information. Examples of the context of the user include a position of the user, an environment (situation) surrounding the user such as a temperature and humidity or weather, a state of the user such as a posture (sitting, standing, or sleeping) of the user, an action of the user such as a motion (running, walking, or eating) of the user, contents of an utterance by the user, an analysis result of input information such as information of an application that is presently running, and a situation (being occupied, playing a game, eating, or moving) of the user and a duration of the situation. Note that being occupied refers to a situation where the user is being occupied with studies, work, driving, cooking, or the like.

The context processing unit 52 stores an estimation result of the present context of the user and supplies the estimation result to the affect processing unit 54 and the support control unit 55. The context processing unit 52 predicts a future context of the user based on a time sequence of stored context estimation results and obtains a prediction result. The context processing unit 52 supplies the prediction result of a future context of the user to the affect processing unit 54 and the support control unit 55.

The analyzing unit 53 analyses contents of inputs from the user based on the pieces of input information input from the various I/Fs 41. For example, the analyzing unit 53 analyzes contents of an utterance by the user based on input information input from the various I/Fs 41 of the microphone. The analyzing unit 53 analyses contents of a character input by the user based on input information input from the various I/Fs 41 of the keyboard. The analyzing unit 53 analyses contents selected by the user by a touch input based on input information input from the various I/Fs 41 of the touch sensor. The analyzing unit 53 supplies an analysis result of the input information to the context processing unit 52 and the support control unit 55.

The affect processing unit 54 obtains an estimation result of the present affect of the user using a feature amount of the biosignal supplied from the biological processing unit 51. Specifically, based on the feature amount of the biosignal, the affect processing unit 54 represents the estimation result of the present affect of the user according to Russell's circumplex model of affect. Russell's circumplex model of affect is a model that represents emotions in a toric shape using a two-dimensional plane with an abscissa representing valence and an ordinate representing arousal, and the estimation result of the affect can be represented by coordinates on the two-dimensional plane.

In addition, the affect processing unit 54 predicts, with reliability, an affect of the user in a short period of time in the future (for example, an hour or so from now) based on at least one of the estimation result of the affect and the estimation result and the prediction result of the context which are supplied from the context processing unit 52. Specifically, the affect processing unit 54 obtains one or more prediction results of a future affect of the user in Russell's circumplex model of affect and a reliability of each prediction result based on at least one of the estimation result of the affect and the estimation result and the prediction result of the context.

For example, when the user is sitting on a sofa in a living room, arousal of the user is predicted to decline in the future. Therefore, when the estimation result of the context is a state where the user is sitting on the sofa in the living room, for example, the affect processing unit 54 sets the reliability with respect to a prediction result with lower arousal than the estimation result of the present affect to be higher than the reliability with respect to a prediction result with higher arousal.

While arousal of the user is predicted to rise immediately after the user starts working, arousal is predicted to gradually decline as the duration of work becomes longer. Therefore, when the estimation result of the context is a state where work is in progress and the duration of the state exceeds a predetermined time, for example, the affect processing unit 54 sets the reliability of each prediction result in accordance with a period of time by which the predetermined time had been exceed in such a manner that the reliability with respect to a prediction result with lower arousal than the estimation result of the present affect increases while the reliability with respect to a prediction result with higher arousal decreases.

When the user is working, a possibility of a sudden change in the future affect is predicted to be high. Therefore, when the estimation result of the context is that work is in progress, for example, the affect processing unit 54 sets the reliability with respect to a prediction result of an affect that differs from the estimation result of the present affect to be high. On the other hand, when the user is at leisure, it is predicted that the possibility that the present affect is to continue is high. Therefore, when the estimation result of the estimation result is that the user is at leisure, for example, the affect processing unit 54 sets the reliability with respect to a prediction result of a same affect as the estimation result of the present affect to be high.

Note that the affect processing unit 54 may correct an estimation result of an affect based on at least one of an estimation result and a prediction result of a context. For example, when the estimation result of a context is a state where the user is sitting on the sofa in the living room, the affect processing unit 54 performs a correction to lower the arousal of the estimation result of the affect.

The affect processing unit 54 supplies the estimation result and the prediction result with reliability of the context to the support control unit 55.

The support control unit 55 is constituted of a setting unit 61, a determining unit 62, and a result processing unit 63. The support control unit 55 receives input of the estimation result and the prediction result of an affect from the affect processing unit 54 and receives input of an estimation result and a prediction result of a context from the context processing unit 52.

Specifically, the setting unit 61 of the support control unit 55 sets a support content that is a content of support with respect to the user based on an estimation result of at least one of the affect and the context and a prediction result of at least one of the affect and the context. The setting unit 61 supplies the set support content to the supporting unit 57.

The determining unit 62 refers to the database 56 and determines one support method among support methods corresponding to the support content set by the setting unit 61 as a current support method based on an estimation result of at least one of the affect and the context.

In addition, the determining unit 62 refers to the database 56 and determines one support means corresponding to the current support method as current support means based on an estimation result of at least one of the affect and the context. The determining unit 62 supplies the determined support method and support means to the supporting unit 57.

The result processing unit 63 generates a support result with respect to the user based on an estimation result of at least one of the affect and the context of the user before and after the support and the analysis result of the input information supplied from the analyzing unit 53. Specifically, the result processing unit 63 interprets the analysis result of the input information as a feedback from the user with respect to the support. The result processing unit 63 adopts the estimation result of at least one of the affect and the context of the user before and after the support and the feedback from the user as a support result with respect to the user. The result processing unit 63 supplies the database 56 (storage unit) with the support result and causes the database 56 (storage unit) to store the support result as a support result table in association with the support content, the support method, and the support means of the support when the support had been received. The support result table is used when the determining unit 62 determines the support method and the support means. This makes it possible to perform optimum support to each individual user.

The database 56 stores, in advance, a support method table which associates an expected support method and an estimation result of an affect and a context of the user for which support according to the support method is suitable for each support content. The database 56 stores, in advance, a support means table which associates expected support means and an estimation result of an affect and a context of the user for which support according to the support means is suitable for each support method.

In addition, the database 56 stores a support result table which associates the support result supplied from the result processing unit 63 and the support content, the support method, and the support means of the support when the support result had been obtained.

The supporting unit 57 supports the user by audio according to the support content supplied from the setting unit 61 and the support method and the support means supplied from the determining unit 62. Specifically, the supporting unit 57 generates control information for controlling the speaker as the human interface device 13 in such a manner that audio of contents of an utterance corresponding to the support content, the support method, and the support means is output from the speaker. In addition, the supporting unit 57 supplies the control information to the various I/Fs 41 of the speaker. Accordingly, audio of the contents of the utterance corresponding to the support content, the support method, and the support means is output from the speaker and user support by audio is performed.

Instead of providing the database 56 inside the audio agent apparatus 14, the database 56 may be provided outside the audio agent apparatus 14 and connected to the support control unit 55 via a wired or wireless network. For example, the IoT device 12 of the audio support system 10 is installed in a living room of the user.

### Description of Russell's Circumplex Model of Affect

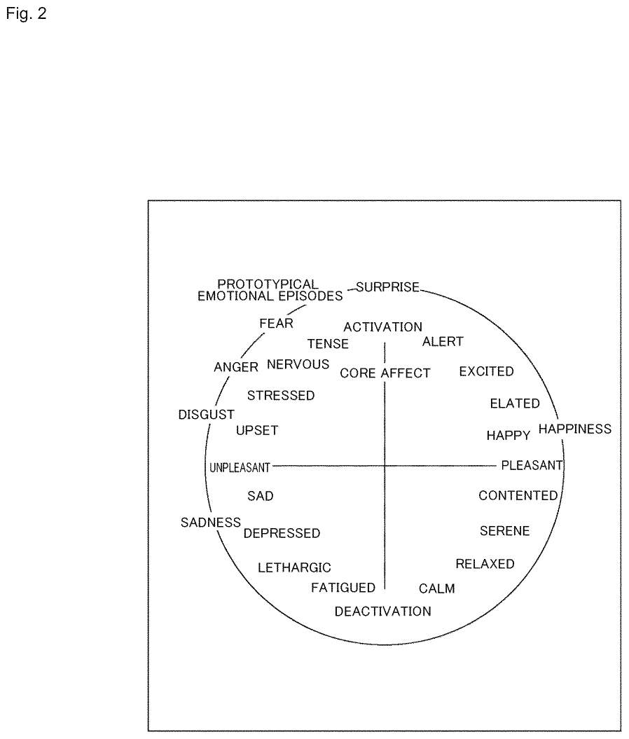

FIG. 2 is a diagram illustrating Russell's circumplex model of affect.

As illustrated in FIG. 2, Russell's circumplex model of affect is a model that represents various emotions in a toric shape using a two-dimensional plane with an abscissa representing valence (pleasant-unpleasant) and an ordinate representing arousal (activation-deactivation).

For example, an emotion of being “tense” can be represented by coordinates which are unpleasant or, in other words, low in valence and high in arousal. An emotion of being “contented” can be represented by coordinates which are high in valence and low in arousal.

As described above, in Russell's circumplex model of affect, various emotions can be represented using a two-dimensional plane with an abscissa representing valence and an ordinate representing arousal. Therefore, using Russell's circumplex model of affect, the affect processing unit 54 indicates an estimation result of the present affect of the user and a prediction result of a future affect of the user with coordinates on a two-dimensional plane.

For example, when the feature amount of a biosignal is a low frequency wave and a high-frequency wave of a heart rate, the affect processing unit 54 recognizes a present state of sympathetic nerves of the user based on the low-frequency wave and the high-frequency wave. In addition, for example, when a state of the sympathetic nerves of the user is a state where the sympathetic nerves are intensely active, the affect processing unit 54 determines a value of an ordinate corresponding to arousal of the estimation result of the present affect of the user to be a high value. When the feature amount of the biosignal is a θ wave of a brain wave, the affect processing unit 54 recognizes a present degree of concentration of the user based on the θ wave. In addition, for example, when the degree of concentration of the user is high, the affect processing unit 54 determines a value of an ordinate corresponding to arousal of the estimation result of the present affect of the user to be a high value.

### Example of Database

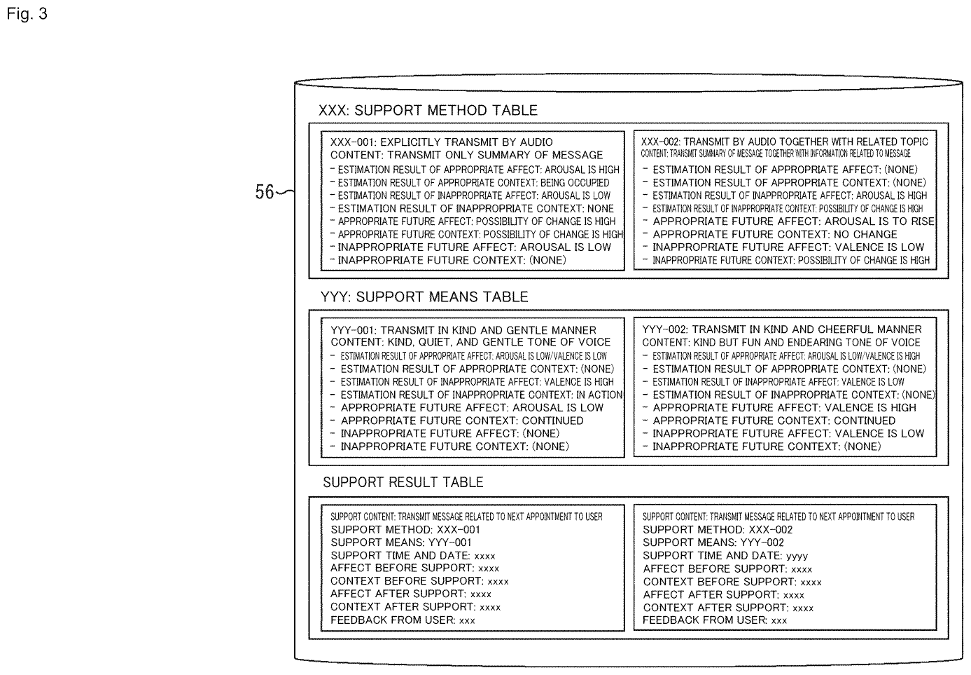

FIG. 3 is a diagram illustrating an example of information stored in the database 56 illustrated in FIG. 1.

FIG. 3 illustrates a support method table corresponding to a support content “transmit message related to next appointment to user,” a support means table corresponding to a support method represented by information including “transmit by audio,” and a support result table which are stored in the database 56.

Specifically, in the example illustrated in FIG. 3, in the support method table corresponding to a support content “transmit message related to next appointment to user,” “arousal is high” representing an estimation result of an appropriate affect, “being occupied” representing an estimation result of an appropriate context, “arousal is low” representing an estimation result of an inappropriate affect, “none” representing an estimation result of an inappropriate context, “possibility of change is high” representing an appropriate future affect, “possibility of change is high” representing an appropriate future context, “arousal is low” representing an inappropriate future affect, and “(none)” representing an inappropriate future context are registered in association with “explicitly transmit by audio” representing a support method. Note that the support method represented by “explicitly transmit by audio” is a transmission method (delivery method) of transmitting (delivering) only a summary of a message related to a next appointment to the user by audio.

In addition, in association with “transmit by audio together with related topic” representing a support method, “(none)” representing an estimation result of an appropriate affect, “(none)” representing an estimation result of an appropriate context, “arousal is high” representing an estimation result of an inappropriate affect, “possibility of change is high” representing an estimation result of an inappropriate context, “arousal is to rise” representing an appropriate future affect, “no change” representing an appropriate future context, “valence is low” representing an inappropriate future affect, and “possibility of change is high” representing an inappropriate future context are registered. Note that the support method represented by “transmit by audio together with related topic” is a support method in which a summary of a message related to a next appointment is transmitted together with information related to the message.

In the support means table corresponding to a support method represented by information including “transmit by audio,” “arousal is low/valence is low” representing an estimation result of an appropriate affect, “(none)” representing an estimation result of an appropriate context, “valence is high” representing an estimation result of an inappropriate affect, “in action” representing an estimation result of an inappropriate context, “arousal is low” representing an appropriate future affect, “continued” representing an appropriate future context, “(none)” representing an inappropriate future affect, and “(none)” representing an inappropriate future context are registered in association with “transmit in kind and gentle manner” representing support means. Note that the support means represented by “transmit in kind and gentle manner” refers to transmission means of a kind, quiet, and gentle tone of voice.

In addition, in association with support means “transmit in kind and cheerful manner,” “arousal is low/valence is high” representing an estimation result of an appropriate affect, “(none)” representing an estimation result of an appropriate context, “valence is low” representing an estimation result of an inappropriate affect, “(none)” representing an estimation result of an inappropriate context, “valence is high” representing an appropriate future affect, “continued” representing an appropriate future context, “valence is low” representing an inappropriate future affect, and “(none)” representing an inappropriate future context are registered. Note that the support means represented by “transmit in kind and cheerful manner” refers to transmission means of a kind but fun and endearing tone of voice.

In the support result table, as past support history, registered in association with a support content “transmit message related to next appointment to user,” “XXX-001” representing a support method, and “YYY-001” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

In this case, a unique number is attached to each support method in the support method table and the support method represented by “XXX-001” is a support method of which the number is “XXX-001.” In the example illustrated in FIG. 3, the number “XXX-001” is attached to the support method represented by “explicitly transmit by audio.” In a similar manner, support means represented by “YYY-001” is support means of which a unique number attached to each support means in the support means table is “YYY-001.” In the example illustrated in FIG. 3, the number “YYY-001” is attached to the support means represented by “transmit in kind and gentle manner.”

In addition, in the support result table, in association with the support content “transmit message related to next appointment to user,” “XXX-002” representing a support method, and “YYY-002 representing support means, a support time and date at which support embodying the support content had been performed by the support method and the support means, an estimation result of an affect before the support, an estimation result of a context before the support, an estimation result of an affect after the support, an estimation result of a context after the support, and a feedback from the user are registered.

Note that in the example illustrated in FIG. 3, the number “XXX-002” is attached to the support method represented by “transmit by audio together with related topic,” and the support method is the support method represented by “XXX-002.” The number “YYY-002” is attached to the support means represented by the support means “transmit in kind and cheerful manner” and the support means is the support means represented by “YYY-002.”

When arousal of the estimation result of an affect after the support is higher than arousal of the estimation result of the affect before the support as registered in the support result table, the determining unit 62 can determine that the performed support had attracted the user's attention. When valence of the estimation result of an affect after the support is lower than valence of the estimation result of the affect before the support, the determining unit 62 can determine that the performed support had been offensive to the user.

In addition, in the support result table, when an estimation result of a context before and after support which is registered in association with a support content “transmit message that warns of danger while walking to user” does not change, the determining unit 62 can determine that the perform support is not sufficient. In this case, the determining unit 62 determines next support means to be support means that differs from the current support means such as support means “high volume.”

As described above, by generating a support result table, the audio agent apparatus 14 can learn a support method and support means suitable for the user based on estimation results of an affect and a context before and after support. The learning is more efficient than learning solely based on explicit feedback that is input from the user.

Note that the support method table, the support means table, and the support result table stored in the database 56 are not limited to the example illustrated in FIG. 3. For example, “at leisure” representing an estimation result of an appropriate context may be registered in the support method table in association with “transmit by audio together with related topic” which is represented by the support method. Accordingly, when the user is at leisure or, in other words, when there is a high possibility that the present affect is to continue, the support method represented by “transmit by audio together with related topic” is determined as the current support method and the user can engage in a conversation with high continuity.

### Example of Outline of Processing by Audio Support System

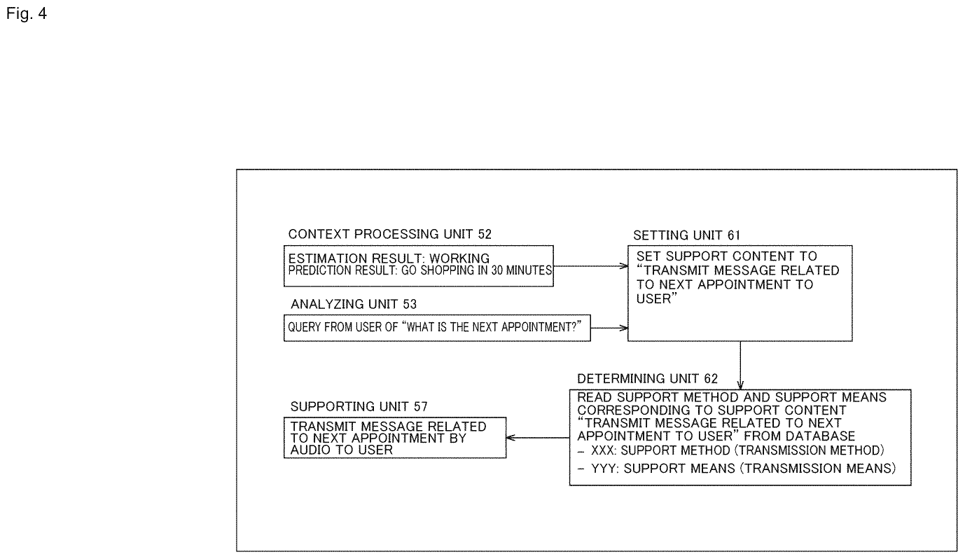

FIG. 4 is a diagram illustrating an example of an outline of processing by the audio support system 10 illustrated in FIG. 1.

As illustrated in FIG. 4, for example, when an estimation result of a context by the context processing unit 52 is “working” and a prediction result of a context with high reliability is “go shopping in 30 minutes” or when an analysis result by the analyzing unit 53 is a query from the user of “What is the next appointment?”, the setting unit 61 sets a support content to “transmit message related to next appointment to user.”

The determining unit 62 selects and reads one support method among support methods corresponding to at least one of an estimation result of an affect and an estimation result of a context from the support method table corresponding to the support content “transmit message related to next appointment to user” stored in the database 56. At this point, based on the support result table, the determining unit 62 selects a support method of which a support result registered in association with the support method is most desirable among the support methods that are selection candidates.

For example, when the estimation result of a context is “working,” the determining unit 62 adopts the support method represented by “explicitly transmit by audio” with the number “XXX-001” which is associated with “working” representing an estimation result of an appropriate context from the support method table illustrated in FIG. 3 as a selection candidate. In addition, when an estimation result of an affect or a context after support corresponding to the number “XXX-001” in the support result table illustrated in FIG. 3 is an appropriate future affect or context corresponding to the number “XXX-001” in the support method table, the determining unit 62 determines the selection candidate as a current support method and reads the selection candidate. As a result, when the user is working or, in other words, when a possibility of a sudden change to a future affect of the user is high, a message related to a next appointment is explicitly transmitted to the user and the user can engage in conversation that emphasizes instantaneity.

In addition, the determining unit 62 selects and reads one support means among support means corresponding to at least one of an estimation result of an affect and an estimation result of a context from the support means table corresponding to the current support method which is stored in the database 56. At this point, based on the support result table, the determining unit 62 selects support means of which a support result registered in association with the support means is most desirable among the support means that are selection candidates.

For example, when valence of the estimation result of an affect is low, the determining unit 62 adopts support means “kind, quiet, and gentle tone of voice” with the number “YYY-001” which is associated with “valence is low” representing an estimation result of an appropriate affect as a selection candidate from the support means table illustrated in FIG. 3. In addition, when the estimation result of an affect or a context after support corresponding to the number “YYY-001” in the support result table illustrated in FIG. 3 is an appropriate future affect or context corresponding to the number “YYY-001” in the support means table, the determining unit 62 determines the selection candidate as current support means and reads the selection candidate.

Based on the support content set by the setting unit 61 and the support method and the support means determined by the determining unit 62, the supporting unit 57 performs support of transmitting a message related to a next appointment by audio to the user. Specifically, the supporting unit 57 generates control information for controlling the speaker so as to output a message related to a next appointment based on the support content, the support method, and the support means and supplies the control information to the various I/Fs 41 of the speaker. For example, when the support method represented by “explicitly transmit by audio” is determined as the current support method and the support means “kind, quiet, and gentle tone of voice” is determined as the current support means, the supporting unit 57 generates control information for controlling the speaker so as to output only a summary of a message related to a next appointment in a kind, quiet, and gentle tone of voice and supplies the control information to the various I/Fs 41 of the speaker. Accordingly, audio that transmits only a summary of a message related to a next appointment in a kind, quiet, and gentle tone of voice is output from the speaker.

The audio that only transmits a summary of the message related to a next appointment is audio that explicitly transmits a next appointment such as “There is an appointment to go shopping with your wife.” On the other hand, audio that transmits a summary of the message related to a next appointment to the user according to the support method represented by “transmit by audio together with related topic” is audio that transmits both the next appointment and information related to the next appointment such as “There is an appointment to go shopping with your wife. The forecast calls for rain. You're low on gas.”

After support by the supporting unit 57, the result processing unit 63 generates a support result based on an estimation result of at least one of an affect and a context of the user before and after the support and an analysis result of input information supplied from the analyzing unit 53. In addition, the result processing unit 63 registers the support result in the support result table in the database 56 in association with the current support content, the current support method, and the current support means. For example, when the current support method is a support method represented by “explicitly transmit by audio” and the current support means is the support means “kind, quiet, and gentle tone of voice,” a leftmost piece of information in the support result table illustrated in FIG. 3 is registered.

### Description of Processing of Audio Agent Apparatus

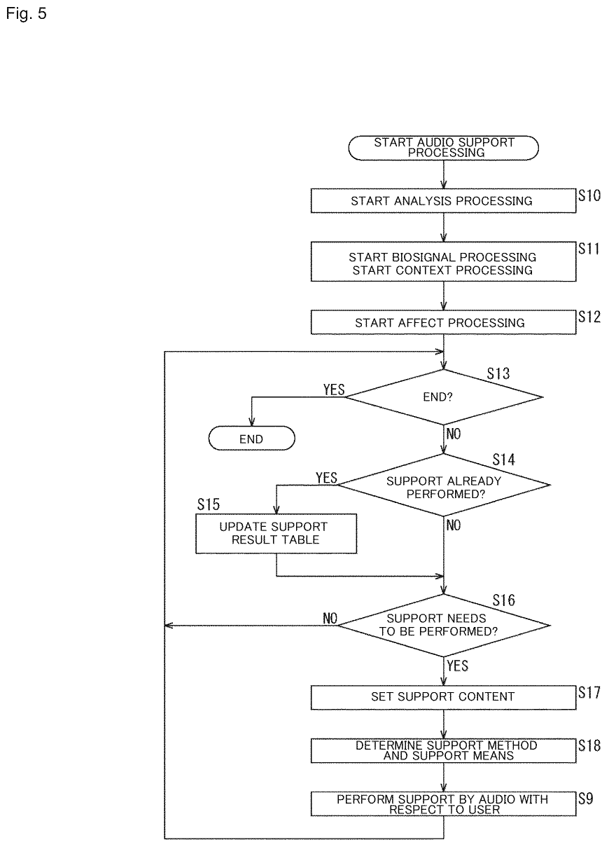

FIG. 5 is a flowchart for describing audio support processing by the audio agent apparatus 14 illustrated in FIG. 1. The audio support processing is started when, for example, an instruction to start the audio support system 10 is issued.

In step S10 in FIG. 5, the analyzing unit 53 of the audio agent apparatus 14 starts analysis processing of analyzing a content of input from the user based on input information that is input from the various I/Fs 41.

In step S11, the biological processing unit 51 starts biosignal processing with respect to a biosignal that is input from the biometric sensor 21. Biosignal processing refers to processing of removing noise in the biosignal based on associated biological information that is input from the motion sensor 22 and extracting various feature amounts from the biosignal after noise removal.

In addition, the context processing unit 52 starts context processing of obtaining an estimation result and a prediction result of a context based on at least one of environmental information that is input from the environmental sensor 31 and a result of the analysis processing started in step S10.

In step S12, the affect processing unit 54 starts affect processing of obtaining an estimation result and a prediction result of an affect. In affect processing, the estimation result of an affect is obtained using the feature amount obtained by the biosignal processing started in step S11. In addition, the prediction result of the affect is obtained based on at least one of the estimation result of the affect and the estimation result and the prediction result of the context obtained by the context processing started in step S11.

In step S13, the audio agent apparatus 14 determines whether or not to end the audio support processing. For example, when a result of the analysis processing is a content indicating the end of the audio support processing, the audio agent apparatus 14 determines to end the audio support processing. When it is determined in step S13 that the audio support processing is to be ended, the audio agent apparatus 14 ends the analysis processing, the biosignal processing, the context processing, and the affect processing and the audio support processing ends.

On the other hand, when it is determined in step S13 that the audio support processing is not to be ended, the processing advances to step S14.

In step S14, the support control unit 55 determines whether support by the supporting unit 57 has already been performed. When it is determined in step S14 that support by the supporting unit 57 has already been performed, the processing advances to step S15.

In step S15, the result processing unit 63 of the support control unit 55 updates the support result table in the database 56. A support result generated based on at least one of an estimation result of a context before and after the support as obtained by context processing and an estimation result of an affect before and after the support as obtained by affect processing and on an analysis result due to analysis processing are used to update the support result table. Subsequently, processing advances to step S16.

On the other hand, when it is determined in step S14 that support has not yet been performed, the processing advances to step S16.

In step S16, the setting unit 61 determines whether or not support needs to be performed with respect to the user based on an estimation result of at least one of an affect and a context and a prediction result of at least one of the affect and the context. When it is determined in step S16 that support needs to be performed with respect to the user, the processing advances to step S17. In step S17, the setting unit 61 sets the support content based on an estimation result of at least one of an affect and a context and a prediction result of at least one of the affect and the context.

In step S18, the determining unit 62 refers to the database 56 and determines the current support method and the current support means based on at least one of an estimation result of an affect and an estimation result of a context and on the support content set in step S17.

In step S19, the supporting unit 57 performs support by audio with respect to the user by generating control information according to the support content set in step S17 and the support method and the support means determined in step S18 and supplying the control information to the various I/Fs 41 of the speaker. Subsequently, the processing returns to step S13 and processing of step S13 and thereafter is performed.

On the other hand, when it is determined in step S16 that support need not be performed, support is not performed, the processing returns to step S13, and processing of step S13 and thereafter is performed.

As described above, the audio agent apparatus 14 supports a user based on an estimation result of at least one of an affect and a context and a prediction result of at least one of the affect and the context. Therefore, the user can be supported by predicting a future state of the user. In addition, the audio agent apparatus 14 determines a support method and support means by referring to a support result table. Therefore, support with respect to the user can be personalized (optimized to the individual).

### Second Embodiment

### Configuration Example of Bicycle Navigation System

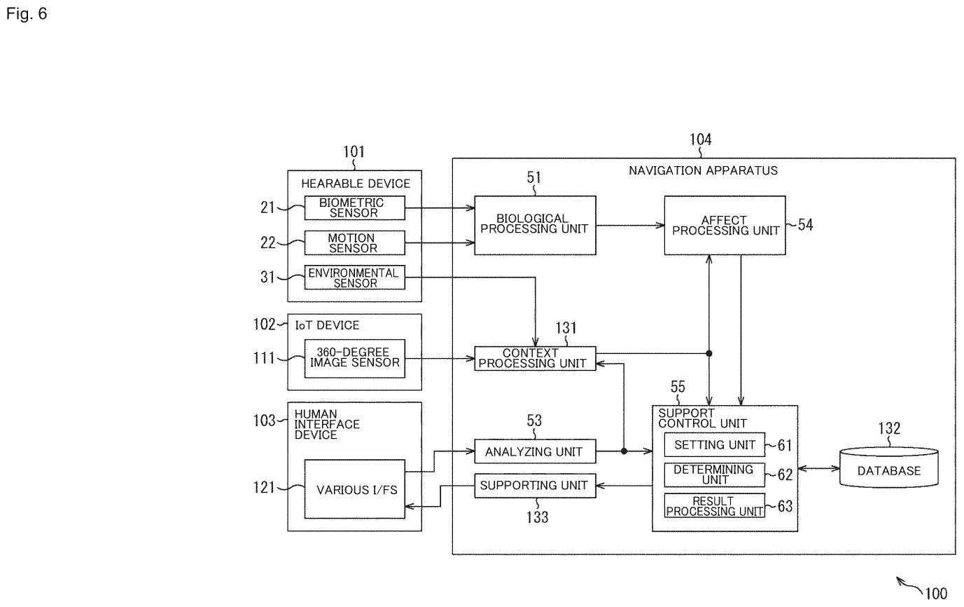

FIG. 6 is a block diagram illustrating a configuration example of a bicycle navigation system including a navigation apparatus as the information processing apparatus to which the present technique has been applied.

In the bicycle navigation system 100 in FIG. 6, portions corresponding to those of the audio support system 10 illustrated in FIG. 1 are denoted by the same reference signs. Therefore, descriptions of such portions will be omitted as appropriate and the description will focus on the portions that differ from the audio support system 10.

The bicycle navigation system 100 is constituted of a hearable device 101, an IoT device 102, one or more human interface devices 103, and a navigation apparatus 104. The bicycle navigation system 100 supports a user riding a bicycle by providing the user with guidance of directions to a destination set by the user and warning of danger in accordance with a degree of urgency or a degree of importance.

Specifically, the hearable device 101 is mounted to an ear of the user. The hearable device 101 differs from the wearable device 11 illustrated in FIG. 1 in that the hearable device 101 is newly provided with an environmental sensor 31 but is otherwise configured in a similar manner to the wearable device 11. Environmental information acquired by the hearable device 101 is input to the navigation apparatus 104.

For example, the IoT device 102 includes a 360-degree image sensor 111 which is installed on a bicycle and which acquires images of surroundings of the user riding the bicycle. An image of the surroundings of the user acquired by the 360-degree image sensor 111 is input as environmental information to the navigation apparatus 104.

The human interface device 103 includes an input device that accepts input from the user and an output device that provides the user with output. Examples of the input device include a microphone, a touch sensor, and a pressure sensor and examples of the output device include earphones and a vibratory apparatus.

The human interface device 103 includes various I/Fs 121 which interact with the user. For example, the various I/Fs 121 of the microphone, the touch sensor, and the pressure sensor input input information to the navigation apparatus 104 in a similar manner to the various I/Fs 41. The various I/Fs 121 of the earphones and the vibratory apparatus provide support by outputting audio and vibrations to the user based on control information input from the navigation apparatus 104.

Information is exchanged between the hearable device 101, the IoT device 102, and the human interface device 103 and the navigation apparatus 104 via a wired or wireless network.

The navigation apparatus 104 differs from the audio agent apparatus 14 in that the navigation apparatus 104 is provided with the context processing unit 131, the database 132, and the supporting unit 133 in place of the context processing unit 52, the database 56, and the supporting unit 57 but is otherwise configured in a similar manner to the audio agent apparatus 14.

The context processing unit 131 of the navigation apparatus 104 differs from the context processing unit 52 illustrated in FIG. 1 in that environmental information used to estimate a context is input from at least one of the environmental sensor 31 and the 360-degree image sensor 111 but is otherwise configured in a similar manner to the context processing unit 52.

While the database 132 stores a support method table, a support means table, and a support result table in a similar manner to the database 56, the support method table and the support means table are suitable for support by the navigation apparatus 104 and the support result table corresponds to support by the navigation apparatus 104.

The supporting unit 133 supports the user by audio or vibration according to the support content supplied from the setting unit 61 and the support method and the support means supplied from the determining unit 62.

Specifically, the supporting unit 133 generates control information for controlling the earphones as the human interface device 103 in such a manner that audio of contents of an utterance corresponding to the support content, the support method, and the support means is output from the earphones. In addition, the supporting unit 133 supplies the control information to the various I/Fs 121 of the earphones. Accordingly, audio of the content of an utterance corresponding to the support content, the support method, and the support means is output from the earphones and user support by audio is performed.

In addition, the supporting unit 133 generates control information for controlling the vibratory apparatus as the human interface device 103 in such a manner that vibration corresponding to the support content, the support method, and the support means is output from the vibratory apparatus. Furthermore, the supporting unit 133 supplies the control information to the various I/Fs 121 of the vibratory apparatus. Accordingly, vibration corresponding to the support content, the support method, and the support means is output from the vibratory apparatus and user support by vibration is performed.

Note that the hearable device 101 and the human interface device 103 may be integrated. Instead of providing the database 132 inside the navigation apparatus 104, the database 132 may be provided outside the navigation apparatus 104 and connected to the support control unit 55 via a wired or wireless network.

### Example of Database

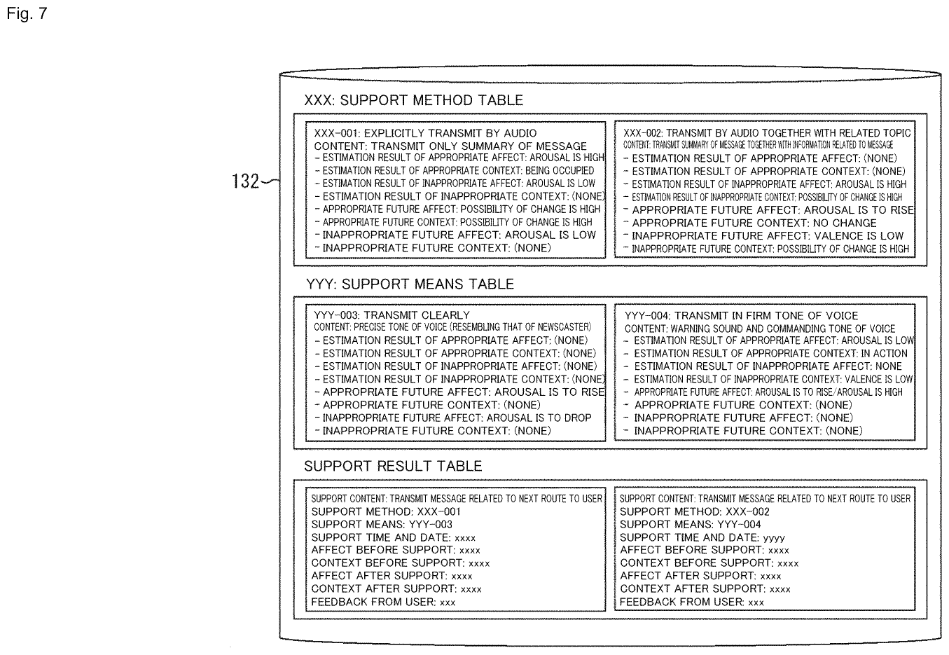

FIG. 7 is a diagram illustrating an example of information stored in the database 132 illustrated in FIG. 6.

FIG. 7 illustrates a support method table corresponding to a support content “transmit message related to next route to user,” a support means table corresponding to a support method represented by information including “transmit by audio,” and a support result table which are stored in the database 132.

Specifically, in the example illustrated in FIG. 7, in the support method table corresponding to the support content “transmit message related to next route to user,” support methods with the number “XXX-001” and the number “XXX-002” in FIG. 3 are registered.

In the support means table corresponding to a support method represented by information including “transmit by audio,” “(none)” representing an estimation result of an appropriate affect, “(none)” representing an estimation result of an appropriate context, “(none)” representing an estimation result of an inappropriate affect, “(none)” representing an estimation result of an inappropriate context, “arousal is to rise” representing an appropriate future affect, “(none)” representing an appropriate future context, “arousal is to drop” representing an inappropriate future affect, and “(none)” representing an inappropriate future context are registered in association with “transmit clearly” representing support means. Note that the support means represented by “transmit clearly” is transmission means of a precise tone of voice (a tone of voice resembling that of a newscaster). A number “YYY-003” is attached to this support means.

In addition, in association with “transmit in firm tone of voice” representing support means, “arousal is low” representing an estimation result of an appropriate affect, “in action” representing an estimation result of an appropriate context, “none” representing an estimation result of an inappropriate affect, “valence is low” representing an estimation result of an inappropriate context, “arousal is to rise/arousal is high” representing an appropriate future affect, “(none)” representing an appropriate future context, “(none)” representing an inappropriate future affect, and “(none)” representing an inappropriate future context are registered. Note that the support means represented by “transmit in firm tone of voice” refers to transmission means of a warning sound and a commanding tone of voice. A number “YYY-004” is attached to this support means.

In the support result table, registered in association with a support content “transmit message related to next route to user,” “XXX-001” representing a support method, and “YYY-003” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Registered in association with a support content “transmit message related to next route to user,” “XXX-002” representing a support method, and “YYY-004” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Note that the support method table, the support means table, and the support result table stored in the database 132 are not limited to the example illustrated in FIG. 7.

For example, when support methods with numbers “XXX-001” and “XXX-002” in FIG. 7 are support methods corresponding to a support content “transmit message that warns of danger to user,” a support method “gentle tone of voice” may be registered in association with “arousal has risen” representing an estimation result of an appropriate affect in the support means table corresponding to the support method including “transmit by audio.”

Accordingly, for example, when an estimation result of a context is “there are dangerous goods” and the support content “transmit message that warns of danger to user” has been set, a selection of the support method including “transmit by audio” causes a message that warns of danger to be transmitted to the user in a gentle tone of voice when the arousal of the rises or, in other words, when the user is aware of the danger that is to be warned. As a result, a situation of causing discomfort to the user by issuing a warning in a stern tone of voice with respect to a danger that the user is already aware of.

On the other hand, a support method “high volume” may be registered in association with “arousal is not to rise” representing an estimation result of an appropriate affect. Accordingly, when the arousal of the user is not to rise or, in other words, when it is presumed that the user is not aware of the danger that is to be warned, a message that warns of danger is transmitted to the user in a high volume. As a result, the attention of the user can be attracted to a danger that the user is not aware of to enable the user to avoid the danger.

Note that audio in support of a support content “transmit message that warns of dangerous goods present in front” in accordance with the support method “explicitly transmit by audio” is, for example, audio that explicitly warns that dangerous goods are present in front such as “dangerous goods in front.”

### Example of Outline of Processing by Bicycle Navigation System

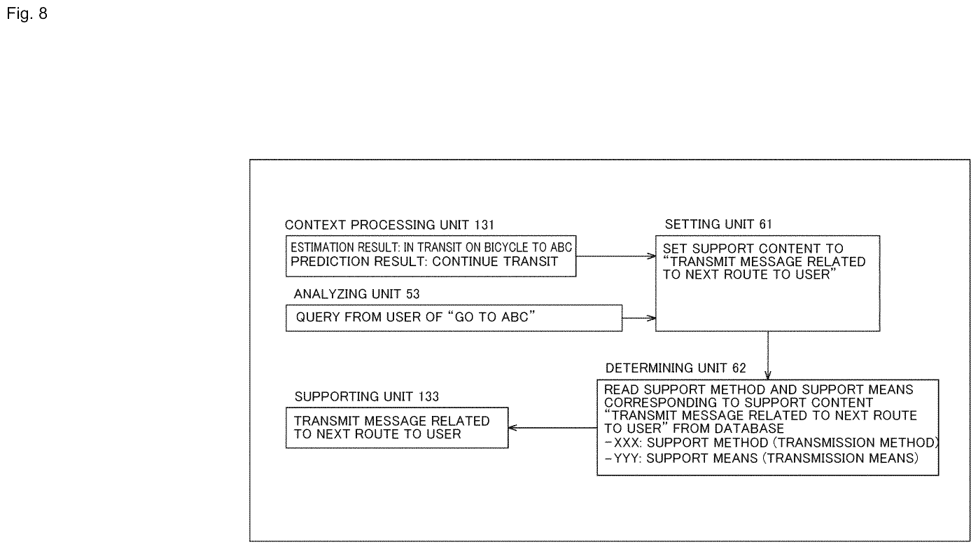

FIG. 8 is a diagram illustrating an example of an outline of processing by the bicycle navigation system 100 illustrated in FIG. 6.

As illustrated in FIG. 8, for example, when an estimation result of a context by the context processing unit 131 is “in transit on bicycle to ABC” and a prediction result of a context with high reliability is “continue transit” or when an analysis result by the analyzing unit 53 is a query from the user of “Go to ABC,” the setting unit 61 sets a support content to “transmit message related to next route to user.”

The determining unit 62 selects and reads one support method among support methods corresponding to at least one of an estimation result of an affect and an estimation result of a context from the support method table corresponding to the support content “transmit message related to next route to user” stored in the database 132. At this point, based on the support result table, the determining unit 62 selects a support method of which a support result registered in association with the support method is most desirable among the support methods that are selection candidates.

For example, when arousal of the estimation result of a context is high, the determining unit 62 adopts the support method represented by “explicitly transmit by audio” with the number “XXX-001” which is associated with “arousal is high” representing an estimation result of an appropriate affect from the support method table illustrated in FIG. 7. In addition, when an estimation result of an affect or a context after support corresponding to the number “XXX-001” in the support result table illustrated in FIG. 7 is an appropriate future affect or context corresponding to the number “XXX-001” in the support method table, the determining unit 62 determines the selection candidate as a current support method and reads the selection candidate.

In addition, the determining unit 62 selects and reads one support means among support means corresponding to at least one of an estimation result of an affect and an estimation result of a context from the support means table corresponding to the current support method which is stored in the database 132. At this point, based on the support result table, the determining unit 62 selects support means of which a support result registered in association with the support means is most desirable among the support means that are selection candidates.

For example, the determining unit 62 adopts support means “precise tone of voice” with the number “YYY-003” from the support method table illustrated in FIG. 7. In addition, for example, when the estimation result of an affect after support corresponding to the support means “precise tone of voice” in the support result table illustrated in FIG. 7, when the estimation result of an affect or a context after support corresponding to the number “YYY-003” in the support means table is an appropriate future affect or context corresponding to the number “YYY-003” in the support means table, the determining unit 62 determines the selection candidate as current support means and reads the selection candidate.

Based on the support content set by the setting unit 61 and the support method and the support means determined by the determining unit 62, the supporting unit 133 performs support of transmitting a message related to a next route to the user as navigation information. Specifically, the supporting unit 133 generates control information based on the support content, the support method, and the support means and supplies the control information to the various I/Fs 121.

For example, when the support method represented by “explicitly transmit by audio” is determined as the current support method and the support means “precise tone of voice” is determined as the current support means, the supporting unit 133 generates control information for causing the speaker to output only a summary of a message related to a next route in a precise tone of voice and supplies the control information to the various I/Fs 121 of the speaker. Accordingly, audio that transmits only a summary of a message related to a next route in a precise tone of voice is output from the speaker.

The audio that only transmits a summary of the message related to a next route is audio that explicitly transmits a next route such as “After 300 meters, turn right” or “Turn right at next corner.” On the other hand, audio that transmits a summary of the message related to a next route to the user according to the support method represented by “transmit by audio together with related topic” is audio that transmits both the next route and information related to the next route such as “After 300 meters, turn right. There is a traffic jam after that. It is likely to rain.”

After support by the supporting unit 133, the result processing unit 63 generates a support result based on an estimation result of at least one of an affect and a context of the user before and after the support and an analysis result of input information supplied from the analyzing unit 53. In addition, the result processing unit 63 registers the support result in the support result table in the database 132 in association with the current support content, the current support method, and the current support means. For example, when the current support method is a support method represented by “explicitly transmit by audio” and the support means is the support means “precise tone of voice,” a leftmost piece of information in the support result table illustrated in FIG. 7 is registered.

The support result table is to be used when determining a support method and support means of a next support. For example, in the support result table, when arousal of an estimation result of an affect before and after support corresponding to the support content “transmit message related to next route to user,” the number “XXX-001” representing a support method, and the number “YYY-003” representing support means does not change and a speed of a bicycle represented by an estimation result of a context before and after the support also does not change, the estimation results of the affect and the context after the support are not an appropriate future affect and an appropriate future context which correspond to the support method and the support means. In other words, in such a case, there is a possibility that the support which has just been performed did not attract the attention of the user and the user did not accurately receive the message related to the next route. Therefore, for example, the determining unit 62 determines support means that attracts the attention of the user other than the support means with the number “YYY-003” as next support means.

For example, the determining unit 62 determines support means with a number “YYY-004” as the next support means. Accordingly, during the next support, after a warning sound of “ding dong” is output, a message related to a next route is transmitted to the user in a commanding tone of voice. Note that a sound may be output before a message related to a next route during support by the support means with the number “YYY-003” as long as the sound is a sound other than the warning sound.

Alternatively, when support means “high volume” is registered in the support means table, the determining unit 62 selects the support means. Accordingly, a message related to a next route is transmitted to the user in a high volume. Alternatively, when support means “termination that attracts attention” is registered in the support means table, the determining unit 62 selects the support means. Accordingly, a message related to a next route of which a termination differs from an ordinary termination such as “Turn right at next corner, okay?” or “At next corner, turn right. Turn right.” is transmitted to the user.

While a case where the determining unit 62 determines support means using the support result table has been described, a support method can be determined in a similar manner. As described above, by determining a support method and support means using a support result table, the determining unit 62 can ensure that the user receives a message related to a next route.

Since a flow of navigation processing performed by the bicycle navigation system 100 is basically similar to the flow of audio support processing illustrated in FIG. 5, a description will be omitted.

As described above, the navigation apparatus 104 supports the user based on an estimation result of at least one of an affect and a context and a prediction result of at least one of the affect and the context. Therefore, the user can be supported by predicting a future state of the user. In addition, the navigation apparatus 104 determines a support method and support means by referring to a support result table. Therefore, support with respect to the user can be optimized to the individual.

### Third Embodiment

### Configuration Example of Pet-Type Robot System

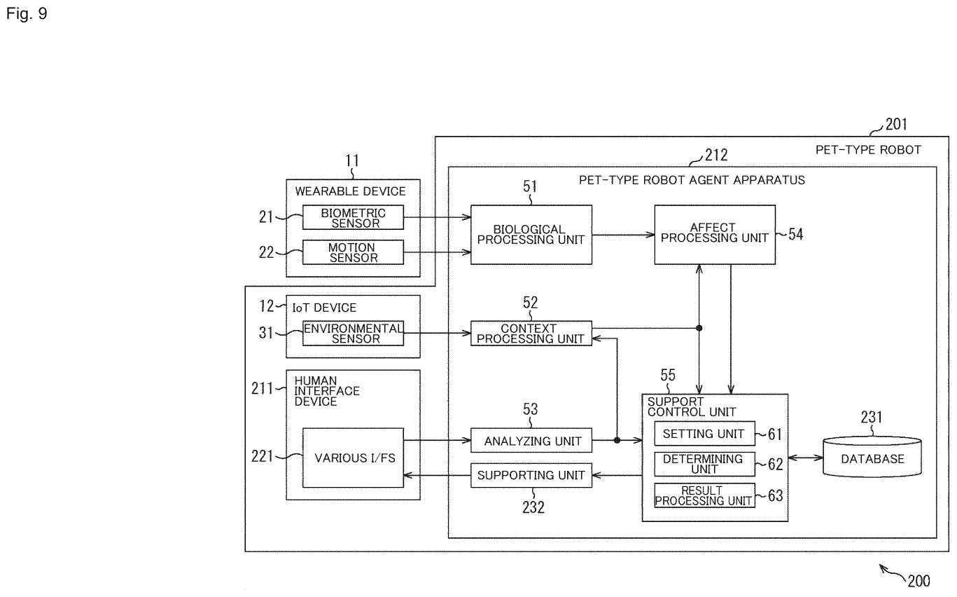

FIG. 9 is a block diagram illustrating a configuration example of a pet-type robot system including a pet-type robot agent apparatus as an information processing apparatus to which the present technique is applied.

In the pet-type robot system 200 illustrated in FIG. 9, portions corresponding to those of the audio support system 10 illustrated in FIG. 1 are denoted by the same reference signs. Therefore, descriptions of such portions will be omitted as appropriate and the description will focus on the portions that differ from the audio support system 10.

The pet-type robot system 200 illustrated in FIG. 9 is constituted of one or more wearable devices 11 and a pet-type robot 201. The pet-type robot system 200 supports the user by managing stress of the user so as to reduce the stress.

Specifically, the pet-type robot 201 of the pet-type robot system 200 includes one or more IoT devices 12, one or more human interface devices 211, and a pet-type robot agent apparatus 212.

The human interface device 211 includes an input device that accepts input from the user and an output device that provides the user with output. Examples of the input device include a microphone as an ear of the pet-type robot 201, a touch sensor, and a pressure sensor and examples of the output device include a speaker as a mouth of the pet-type robot 201 and a driving unit which drives each part (not illustrated) of the pet-type robot.

The human interface device 211 includes various I/Fs 221 which interact with the user. For example, the various I/Fs 221 of the microphone, the touch sensor, and the pressure sensor input input information to the navigation apparatus 104 in a similar manner to the various I/Fs 41. The various I/Fs 221 of the speaker provide support by outputting audio to the user based on control information input from the pet-type robot agent apparatus 212. The various I/Fs 221 of the driving unit provide support by driving each part of the pet-type robot 201 based on control information input from the pet-type robot agent apparatus 212.

Information is exchanged between the wearable device 11 and the pet-type robot 201 via a wired or wireless network.

The pet-type robot agent apparatus 212 differs from the audio agent apparatus 14 in that the pet-type robot agent apparatus 212 is provided with the database 231 and the supporting unit 232 in place of the database 56 and the supporting unit 57 but is otherwise configured in a similar manner to the audio agent apparatus 14.

While the database 231 of the pet-type robot agent apparatus 212 stores a support method table, a support means table, and a support result table in a similar manner to the database 56, the support method table and the support means table are suitable for support by the pet-type robot agent apparatus 212 and the support result table corresponds to support by the pet-type robot agent apparatus 212.

The supporting unit 232 supports the user via the pet-type robot 201 according to the support content supplied from the setting unit 61 and the support method and the support means supplied from the determining unit 62.

Specifically, the supporting unit 232 performs user support by audio of the pet-type robot 201 by generating control information for controlling the speaker and supplying the control information to the various I/Fs 321 of the speaker in a similar manner to the supporting unit 57.

In addition, the supporting unit 232 generates control information for controlling the driving unit so that the pet-type robot 201 makes a motion corresponding to the support content, the support method, and the support means. Furthermore, the supporting unit 232 supplies the control information to the various I/Fs 221 of the driving unit. Accordingly, the pet-type robot 201 makes a predetermined motion and user support by the motion of the pet-type robot 201 is performed.

Note that an image sensor among the IoT devices 12 of the pet-type robot 201 is installed as, for example, an eye of the pet-type robot 201. Instead of providing the database 231 inside the pet-type robot 201, the database 231 may be provided outside the pet-type robot 201 and connected to the support control unit 55 via a wired or wireless network.

### Example of Database

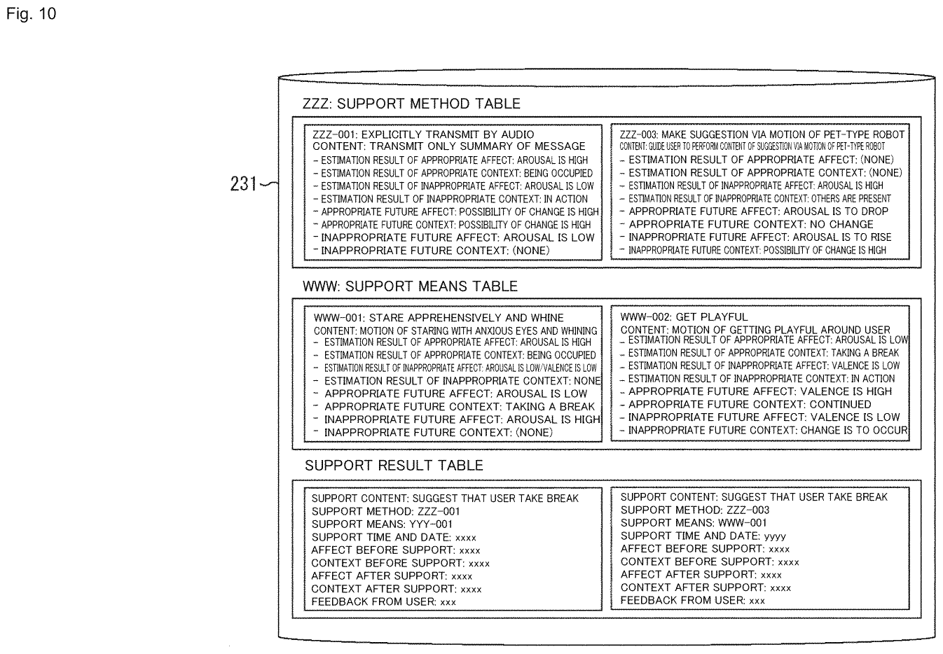

FIG. 10 is a diagram illustrating an example of information stored in the database 231 illustrated in FIG. 9.

FIG. 10 illustrates a support method table corresponding to a support content “suggest that user take break,” a support means table corresponding to a support method “make suggestion via motion of pet-type robot,” and a support result table which are stored in the database 231.

Specifically, in the example illustrated in FIG. 10, in the support method table corresponding to the support content “suggest that user take break,” “arousal is high” representing an estimation result of an appropriate affect, “being occupied” representing an estimation result of an appropriate context, “arousal is low” representing an estimation result of an inappropriate affect, “in action” representing an estimation result of an inappropriate context, “possibility of change is high” representing an appropriate future affect, “possibility of change is high” representing an appropriate future context, “arousal is low” representing an inappropriate future affect, and “(none)” representing an inappropriate future context are registered in association with “explicitly suggest by audio” representing a support method. Note that the support method represented by “explicitly transmit by audio” is a transmission method of transmitting only a summary of a message related to a content of a suggestion by audio.

In association with “make suggestion via motion of pet-type robot” representing a support method, “(none)” representing an estimation result of an appropriate affect, “(none)” representing an estimation result of an appropriate context, “arousal is high” representing an estimation result of an inappropriate affect, “others are present” representing an estimation result of an inappropriate context, “arousal is to drop” representing an appropriate future affect, “no change” representing an appropriate future context, “arousal is to rise” representing an inappropriate future affect, and “possibility of change is high” representing an inappropriate future context are registered. Note that the support method represented by “make suggestion via motion of pet-type robot” is a suggestion method of guiding the user to perform the content of the suggestion via a motion of the pet-type robot 201.

In the support means table corresponding to the support method “make suggestion via motion of pet-type robot,” “arousal is high” representing an estimation result of an appropriate affect, “being occupied” representing an estimation result of an appropriate context, “arousal is low/valence is low” representing an estimation result of an inappropriate affect, “none” representing an estimation result of an inappropriate context, “arousal is low” representing an appropriate future affect, “taking a break” representing an appropriate future context, “arousal is high” representing an inappropriate future affect, and “(none)” representing an inappropriate future context are registered in association with “stare apprehensively and whine” representing support means. Note that the support means represented by “stare apprehensively and whine” is suggestion means that is a motion of staring with anxious eyes and whining. A number “WWW-001” is attached to this support means.

In addition, “arousal is low” representing an estimation result of an appropriate affect, “taking break” representing an estimation result of an appropriate context, “valence is low” representing an estimation result of an inappropriate affect, “in action” representing an estimation result of an inappropriate context, “valence is high” representing an appropriate future affect, “continued” representing an appropriate future context, “valence is low” representing an inappropriate future affect, and “change is to occur” representing an inappropriate future context are registered in association with “get playful” representing support means. Note that the support means represented by “get playful” is suggestion means that is a motion of getting playful around the user. A number “WWW-002” is attached to this support means.

In the support result table, registered in association with a support content “suggest that user take break,” “ZZZ-001” representing a support method, and “YYY-001” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user. In the example illustrated in FIG. 10, support means of the number “YYY-001” in FIG. 3 is registered in the support means table corresponding to the support method including “suggest by audio.”

Registered in association with a support content “suggest that user take break,” “ZZZ-003” representing a support method, and “WWW-001” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Note that the support method table, the support means table, and the support result table stored in the database 231 are not limited to the example illustrated in FIG. 10.

### Example of Outline of Processing by Pet-Type Robot System

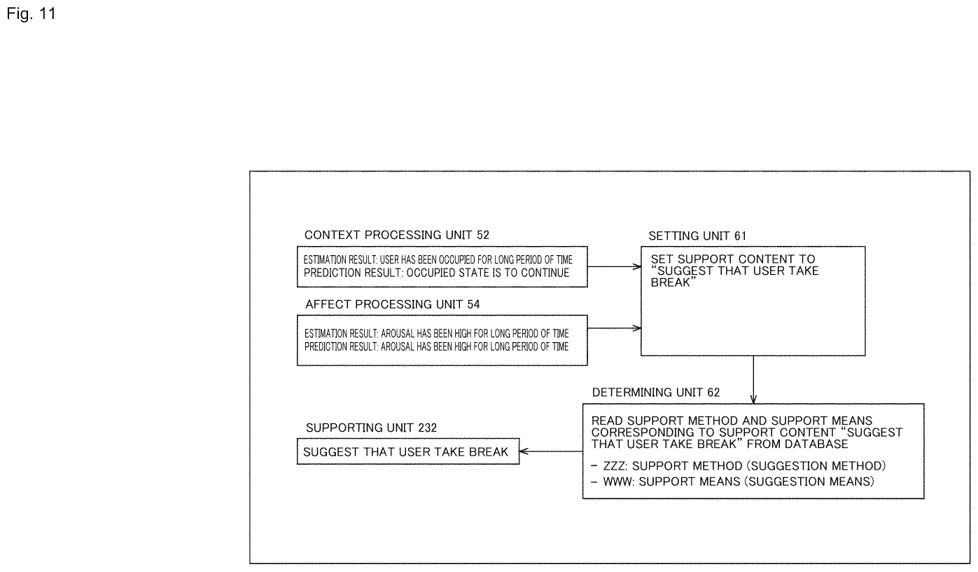

FIG. 11 is a diagram illustrating an example of an outline of processing by the pet-type robot system 200 illustrated in FIG. 9.

As illustrated in FIG. 11, for example, when an estimation result of a context by the context processing unit 52 is “user has been occupied for long period of time” and a prediction result of a context with high reliability is “occupied state is to continue” and an estimation result of an affect and a prediction result with high reliability by the affect processing unit 54 “arousal has been high for long period of time,” the setting unit 61 sets a support content to “suggest that user take break.” In other words, when the user has been occupied in a concentrated manner for a long period of time, since a stress level of the user is high, the setting unit 61 suggests that the user take a break and makes the user relax.

The determining unit 62 selects and reads one support method among support methods corresponding to at least one of an estimation result of an affect and an estimation result of a context from the support method table corresponding to the support content “suggest that user take break” stored in the database 231. At this point, based on the support result table, the determining unit 62 selects a support method of which a support result registered in association with the support method is most desirable among the support methods that are selection candidates.

For example, since arousal of the estimation result of an affect is high, the determining unit 62 adopts the support method represented by “explicitly transmit by audio” with the number “ZZZ-001” which is associated with “arousal is high” representing an estimation result of an appropriate affect from the support method table illustrated in FIG. 10. In addition, when an estimation result of an affect or a context after support corresponding to the number “ZZZ-001” in the support result table illustrated in FIG. 10 is an appropriate future affect or context corresponding to the number “ZZZ-001” in the support method table, the determining unit 62 determines the selection candidate as a current support method and reads the selection candidate.

In addition, the determining unit 62 selects and reads one support means among support means corresponding to at least one of an estimation result of an affect and an estimation result of a context from the support means table corresponding to the current support method which is stored in the database 231. At this point, based on the support result table, the determining unit 62 selects support means of which a support result registered in association with the support means is most desirable among the support means that are selection candidates.

For example, when the support means with the numbers “YYY-001” and “YYY-002” in FIG. 3 are registered as the support means table corresponding to the support method including “suggest by audio” and valence of the estimation result of an affect is low, the determining unit 62 adopts the support means “kind, quiet, and gentle tone of voice” with the number “YYY-001” associated with “valence is low” representing an estimation result of the affect as a selection candidate from the support means table. In addition, when an estimation result of an affect or a context after support corresponding to the number “YYY-001” in the support result table illustrated in FIG. 10 is an appropriate future affect or context corresponding to the number “YYY-001” in the support means table, the determining unit 62 determines the support means as current support means and reads the support means.

Based on the support content set by the setting unit 61 and the support method and the support means determined by the determining unit 62, the supporting unit 232 performs support of suggesting that the user take a break via the pet-type robot 201. Specifically, the supporting unit 232 generates control information based on the support content, the support method, and the support means and supplies the control information to the various I/Fs 221.

For example, when the support method represented by “explicitly transmit by audio” is determined as the current support method and the support means “kind, quiet, and gentle tone of voice” is determined as the current support means, the supporting unit 232 generates control information for causing the speaker to output only a summary of a message related to a suggestion to take a break in a kind, quiet, and gentle tone of voice and supplies the control information to the various I/Fs 221 of the speaker. Accordingly, audio that transmits only a summary of a message related to a suggestion to take a break in a kind, quiet, and gentle tone of voice is output from the speaker. Note that audio that transmits only a summary of a message related to a suggestion to take a break is, for example, audio that explicitly suggests taking a break such as “How about taking a break?”

In addition, when the support method represented by “make suggestion via motion of pet-type robot” is determined as the current support method and the support means “motion of staring with anxious eyes and whining” is determined as the current support means, the supporting unit 232 generates control information for controlling the driving unit in such a manner that the pet-type robot 201 makes a motion of staring with anxious eyes and whining and supplies the control information to the various I/Fs 221 of the driving unit. Furthermore, the supporting unit 232 generates control information so that the speaker outputs whining and supplies the control information to various I/Fs 221 of the speaker. According to the above, the pet-type robot 201 stares with anxious eyes and whines.

In other words, in this case, instead of issuing a command of “Let's take a break,” the supporting unit 232 performs support of suggesting that the user take a break by causing the pet-type robot 201 to make a motion that entices the user to take a break.

After support by the supporting unit 232, the result processing unit 63 generates a support result based on an estimation result of at least one of an affect and a context of the user before and after the support and an analysis result of input information supplied from the analyzing unit 53. In addition, the result processing unit 63 registers the support result in the support result table in the database 231 in association with the current support content, the current support method, and the current support means. For example, when the current support method is a support method represented by “explicitly transmit by audio” and the current support means is the support means “kind, quiet, and gentle tone of voice,” a leftmost piece of information in the support result table illustrated in FIG. 10 is registered.

The support result table is to be used when determining a support method and support means of a next support. For example, in the support result table, when an estimation result of a context after support which corresponds to the support content “suggest that user take break,” the number “ZZZ-001” representing a support method, and the number “YYY-001” representing support means is not “taking break,” the estimation result of the context after support is not an appropriate future context that corresponds to the support method and the support means. In other words, in such a case, there is a possibility that the support which has just been performed did not attract the attention of the user. Therefore, the determining unit 62 determines at least one of a support method and support means of a next support to differ from that of the current support.

For example, the determining unit 62 selects a support method represented by “make suggestion via motion of pet-type robot” as a next support method and selects support means “motion of approaching and throwing itself against user” which is one of the support means corresponding to the support method as next support means. Alternatively, the determining unit 62 selects a support method represented by “make suggestion via motion of pet-type robot” as a next support method and selects support means “motion of singing song suitable for break” which is one of the support means corresponding to the support method as next support means.

On the other hand, when an estimation result of a context and a prediction result with high reliability is “taking break” and arousal of an estimation result of an affect and a prediction result with high reliability is low, the setting unit 61 sets “provide user with enjoyable break” as a support content. When a support method represented by “make suggestion via motion of pet-type robot” is registered in the support method table corresponding to the support content “provide user with enjoyable break,” for example, the determining unit 62 reads support means “get playful” as current support means from the support means table illustrated in FIG. 10. Accordingly, when the user is taking a break and is predicted to continue taking the break and a state of low arousal of the user is predicted to continue, the pet-type robot 201 gets playful with the user.

As described above, the pet-type robot agent apparatus 212 supports the user based on an estimation result of at least one of an affect and a context and a prediction result of at least one of the affect and the context. Therefore, the user can be supported by predicting a future state of the user. In addition, the pet-type robot agent apparatus 212 determines a support method and support means by referring to a support result table. Therefore, support with respect to the user can be optimized to the individual.

### Fourth Embodiment

### Configuration Example of Driving Support System

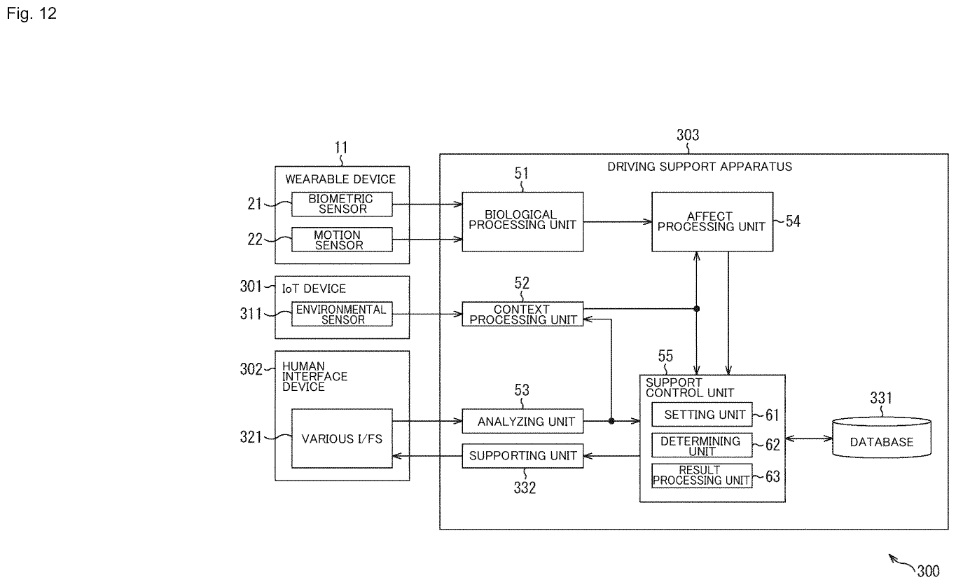

FIG. 12 is a block diagram illustrating a configuration example of a driving support system including a driving support apparatus as an information processing apparatus to which the present technique is applied.

In a driving support system 300 illustrated in FIG. 12, portions corresponding to those of the audio support system 10 illustrated in FIG. 1 are denoted by the same reference signs. Therefore, descriptions of such portions will be omitted as appropriate and the description will focus on the portions that differ from the audio support system 10.

The driving support system 300 illustrated in FIG. 12 is constituted of one or more wearable devices 11, one or more IoT devices 301, one or more human interface devices 302, and a driving support apparatus 303. For example, the driving support system 300 is built into an automobile or the like. The driving support system 300 supports a user driving an automobile or the like so as to enable the user to drive in a comfortable and safe manner.

Specifically, the IoT device 301 includes environmental sensors 311 that are various sensors which acquire environmental information. Examples of the environmental sensors 311 include, in addition to a GPS sensor, an image sensor, an ultrasonic sensor, an infrared camera, an acceleration sensor, a gyroscope sensor, a temperature/humidity sensor, and a weather sensor in a similar manner to the environmental sensors 31, a traffic information acquiring unit or the like which acquires traffic information of the surroundings of the user as environmental information. The IoT device 301 inputs the environmental information acquired by the environmental sensors 311 to the driving support apparatus 303.

The human interface device 302 includes an input device that accepts an input from the user and an output device which provides the user with output. Examples of the input device include a microphone, a touch sensor, and a pressure sensor and examples of the output device include a speaker, a vibratory apparatus, and a display. The human interface device 302 includes various I/Fs 321 that interact with the user. The various I/Fs 321 of the microphone, the touch sensor, and the pressure sensor input input information to the navigation apparatus 104 in a similar manner to the various I/Fs 41. The various I/Fs 321 of the speaker, the vibratory apparatus, and the display provides support by outputting audio, vibration, and video with respect to the user based on control information input from the driving support apparatus 303.

Information exchange between the wearable device 11, the IoT device 301, and the human interface device 302, and the driving support apparatus 303, is performed via a wired or wireless network.

The driving support apparatus 303 differs from the audio agent apparatus 14 in that the driving support apparatus 303 is provided with a database 331 and a supporting unit 332 in place of the database 56 and the supporting unit 57 but is otherwise configured in a similar manner to the audio agent apparatus 14.

While the database 331 of the driving support apparatus 303 stores a support method table, a support means table, and a support result table in a similar manner to the database 56, the support method table and the support means table are suitable for support by the driving support apparatus 303 and the support result table corresponds to support by the driving support apparatus 303.

The supporting unit 332 supports the user by audio, vibration, or video according to the support content supplied from the setting unit 61 and the support method and the support means supplied from the determining unit 62.

Specifically, the supporting unit 332 performs user support by audio by generating control information for controlling the speaker and supplying the control information to the various I/Fs 321 of the speaker in a similar manner to the supporting unit 57. The supporting unit 332 performs user support by vibration by generating control information for controlling the vibratory apparatus and supplying the control information to the various I/Fs 321 of the vibratory apparatus in a similar manner to the supporting unit 133 illustrated in FIG. 6. The supporting unit 332 generates control information for controlling the display as the human interface device 302 so that video corresponding to the support content, the support method, and the support means is displayed on the display. In addition, the supporting unit 332 supplies the control information to the various I/Fs 321 of the display. Accordingly, video corresponding to the support content, the support method, and the support means is displayed on the display and user support by the video is performed.

Note that instead of providing the database 331 inside the driving support apparatus 303, the database 231 may be provided outside the driving support apparatus 303 and connected to the support control unit 55 via a wired or wireless network.

### Example of Database

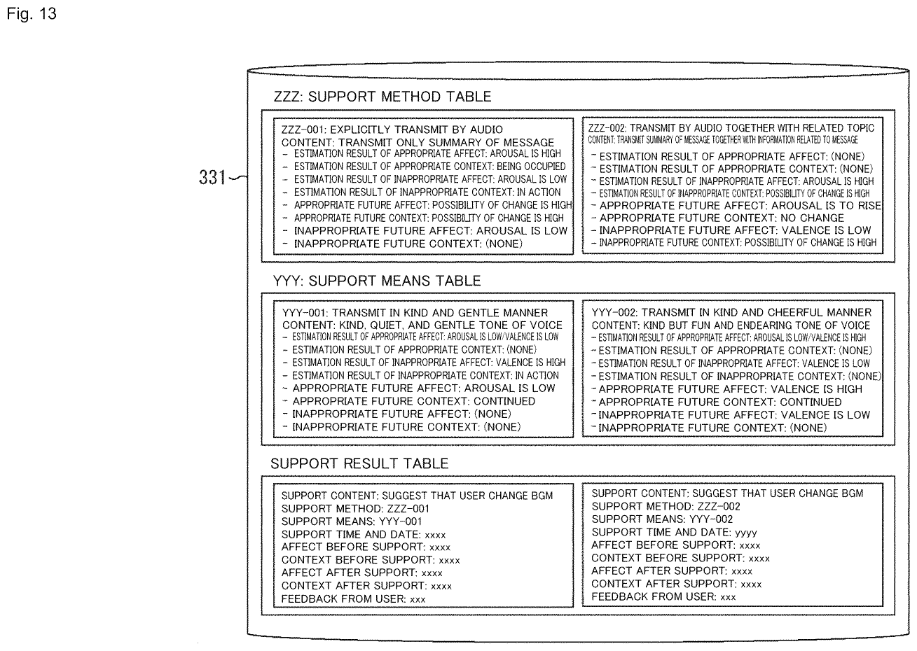

FIG. 13 is a diagram illustrating an example of information stored in the database 331 illustrated in FIG. 12.

FIG. 13 illustrates a support method table corresponding to a support content “suggest that user change BGM (Background Music),” a support means table corresponding to a support method represented by information including “suggest by audio,” and a support result table which are stored in the database 331.

Specifically, in the example illustrated in FIG. 13, in the support method table corresponding to the support content “suggest that user change BGM,” a support method with the number “ZZZ-001” in FIG. 10 is registered.

In association with “suggest by audio together with related topic” representing a support method, “(none)” representing an estimation result of an appropriate affect, “(none)” representing an estimation result of an appropriate context, “arousal is high” representing an estimation result of an inappropriate affect, “possibility of change is high” representing an estimation result of an inappropriate context, “arousal is to rise” representing an appropriate future affect, “no change” representing an appropriate future context, “valence is low” representing an inappropriate future affect, and “possibility of change is high” representing an inappropriate future context are registered. A number “ZZZ-002” is attached to this support method.

Support means with the numbers “YYY-001” and “YYY-002” in FIG. 3 are registered in the support means table corresponding to the support method represented by information including “suggest by audio.”

In the support result table, registered in association with the support content “suggest that user change BGM,” “ZZZ-001” representing a support method, and “YYY-001” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Registered in association with the support content “suggest that user change BGM,” “ZZZ-002” representing a support method, and “YYY-002” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Note that the support method table, the support means table, and the support result table stored in the database 331 are not limited to the example illustrated in FIG. 13.

### Example of Outline of Processing by Driving Support System

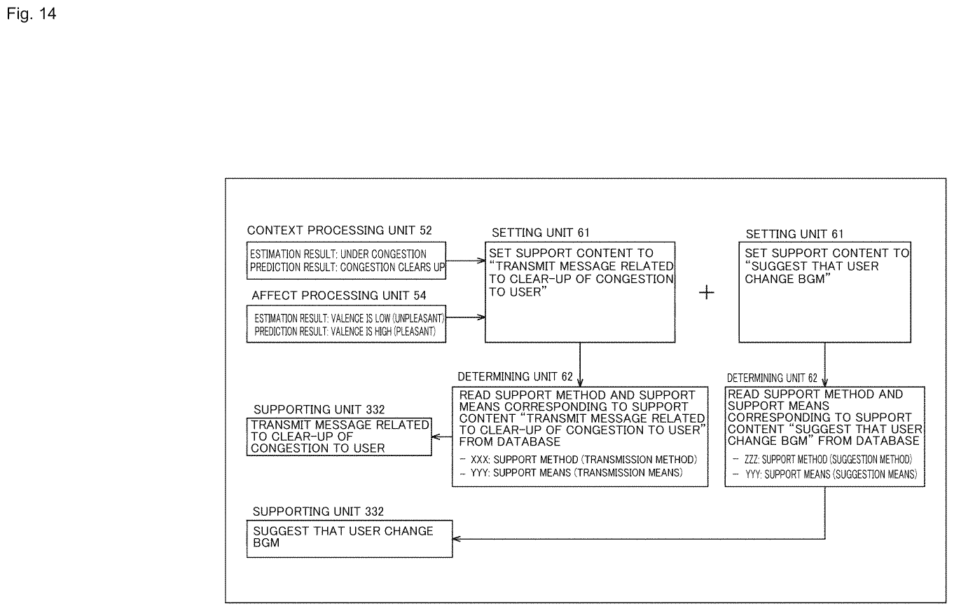

FIG. 14 is a diagram illustrating an example of an outline of processing by the driving support system 300 illustrated in FIG. 12.

As illustrated in FIG. 14, for example, when an estimation result of a context by the context processing unit 52 is “under congestion,” a prediction result of a context with high reliability is “congestion clears up,” valence of an estimation result of an affect by the affect processing unit 54 is low, and a valence of a prediction result of an affect with high reliability is high, the setting unit 61 sets the support content to “transmit message related to clear-up of congestion to user” and “suggest that user change BGM.” In other words, the driving support apparatus 303 provides support of transmitting the clear-up of congestion and suggesting a change in BGM with respect to a user feeling discomfort in a traffic jam and causes a state of the user to transition from a state of low valence to a state of high valence.

The determining unit 62 selects and reads one support method among support methods corresponding to at least one of an estimation result of an affect and an estimation result of a context from the support method table corresponding to the support content “transmit message related to clear-up of congestion to user” stored in the database 331. The determining unit 62 selects and reads one support method among support methods corresponding to at least one of an estimation result of an affect and an estimation result of a context from the support method table corresponding to the support content “suggest that user change BGM” stored in the database 331. At this point, based on the support result tables, the determining unit 62 selects a support method of which a support result registered in association with the support method is most desirable among the support methods that are selection candidates.

For example, when arousal of the estimation result of an affect is high, the determining unit 62 adopts the support method represented by “clearly suggest by audio” with the number “ZZZ-001” which is associated with “arousal is high” representing an estimation result of an appropriate affect from the support method table illustrated in FIG. 13 as a selection candidate. In addition, when an estimation result of an affect or a context after support corresponding to the number “ZZZ-001” in the support result table illustrated in FIG. 13 is an appropriate future affect or context corresponding to the number “ZZZ-001” in the support method table, the determining unit 62 determines the selection candidate as a current support method and reads the selection candidate.

In addition, the determining unit 62 selects and reads one support method among support methods corresponding to at least one of an estimation result of an affect and an estimation result of a context from the support means table corresponding to the current support method which is stored in the database 331. At this point, based on the support result table, the determining unit 62 selects support means of which a support result registered in association with the support means is most desirable among the support means that are selection candidates.

For example, when valence of the estimation result of an affect is low, the determining unit 62 adopts support means “kind, quiet, and gentle tone of voice” with the number “YYY-001” which is associated with “valence is low” representing an estimation result of an affect as a selection candidate from the support means table illustrated in FIG. 13. In addition, when the estimation result of an affect or a context after support corresponding to the number “YYY-001” in the support result table illustrated in FIG. 13 is an appropriate future affect or context corresponding to the number “YYY-001” in the support means table, the determining unit 62 determines the selection candidate as current support means and reads the selection candidate.

Based on the support content set by the setting unit 61 and the support method and the support means determined by the determining unit 62, the supporting unit 332 performs support of transmitting a message related to clear-up of congestion as driving support information for supporting driving by the user and support of suggesting that the user change BGM.

For example, when the support method represented by “clearly transmit by audio” is determined as the current support method and the support means “kind, quiet, and gentle tone of voice” is determined as the current support means with respect to the support content “suggest that user change BGM,” the supporting unit 332 generates control information for controlling the speaker so as to output only a summary of a message related to a suggestion to change the BGM in a kind, quiet, and gentle tone of voice and supplies the control information to the various I/Fs 321 of the speaker. Accordingly, audio that transmits only a summary of a message related to a suggestion to change the BGM in a kind, quiet, and gentle tone of voice is output from the speaker.

The audio that only transmits a summary of a message related to a suggestion to change the BGM is audio that clearly suggests a change in the BGM such as “How about changing the music to ABC (a song with a refreshing tempo)”? On the other hand, audio that transmits a message related to a suggestion to change the BGM to the user according to the support method represented by “transmit by audio together with related topic” is audio that transmits both the suggestion to change the BGM and information related to the suggestion such as “How about changing the music to ABC (a song with a refreshing tempo)? The song put you in a good mood when you went for a drive to BCD a while ago.”

In addition, when the support method with the number “XXX-002” in FIG. 7 is registered in the support method table corresponding to the support content “transmit message related to clear-up of congestion to user,” audio that transmits a message related to clear-up of congestion according to the support method is, for example, audio of “You must be tired by now. The congestion is expected to clear up soon.” In other words, in this case, audio that transmits both the clear-up of congestion and a message appreciating the user's efforts to endure the congestion as information related to the clear-up of congestion is output.

After support by the supporting unit 332, the result processing unit 63 generates a support result based on an estimation result of at least one of an affect and a context of the user before and after the support and an analysis result of input information supplied from the analyzing unit 53. In addition, the result processing unit 63 registers the support result in the support result table in the database 331 in association with the current support content, the current support method, and the current support means. For example, when the current support method is a support method represented by “explicitly transmit by audio” and the current support means is the support means “kind, quiet, and gentle tone of voice,” a leftmost piece of information in the support result table illustrated in FIG. 13 is registered.

The support result table is to be used not only when determining a support method and support means of a next support but also when setting a support content. For example, when arousal of an estimation result of an affect after the current support is low, a present state of the user is a state that is inappropriate for driving. Therefore, the setting unit 61 sets a support content of which arousal of the estimation result of an affect after the support is high as a next support content in the support result table.

For example, the setting unit 61 sets “abruptly transmit traffic information of surroundings to user,” “temporarily change volume of BGM,” “transmit message for checking whether user is awake to user,” or the like as the support content. When audio such as “restore to original volume” or the like is input from the user after support of the support content “temporarily change volume of BGM,” arousal of the estimation result of an affect after the support rises. Therefore, the setting unit 61 sets a next support content without taking into consideration a height of arousal of the estimation result of an affect after support which is registered in association with each support content in the support result table.

On the other hand, when no audio is input from the user after support of the support content “transmit message for checking whether user is awake to user,” arousal of the estimation result of an affect after the support remains low. Therefore, the setting unit 61 sets another support content of which arousal of the estimation result of an affect after the support is high in the support result table such as a support content “transmit message ordering user to wake up to user” as a next support content.

Note that when the arousal of the estimation result of an affect after the support remains low even after performing support corresponding to a plurality of support contents of which arousal of the estimation result of an affect after the support is high in the support result table, there is a possibility of an estimation error of the affect. Therefore, the setting unit 61 sets a support content “transmit message that warns of system error and prompts user to stop car at once” as a next support content.

In the example illustrated in FIG. 13, while the setting unit 61 sets the support content “suggest that user change BGM” in anticipation of acceleration after clear-up of congestion, a support content “suggest measure to avoid congestion to user” or “suggest method of staying comfortable during congestion to user” may be set considering the fact that the congestion is ongoing. Examples of suggesting a measure to avoid congestion include suggesting a route change, taking a break, or making a detour and suggesting stopping at a convenience store or a parking area and purchasing food or beverages. Examples of suggesting a method of staying comfortable during a congestion include suggesting taking turns to drive when the user has been driving for hours and there is another person who can drive, suggesting changing a posture or position to a reclined posture or position, and suggesting taking deep breaths or stretching as far as the user can within the confines of the driver's seat.

When a prediction result of a context is “congestion occurs,” valence of an estimation result of an affect is high, and a prediction result of the affect is “valence is to drop,” for example, the setting unit 61 sets support contents “transmit message related to occurrence of congestion to user” and “suggest measure to avoid congestion to user.” Note that the setting unit 61 may set the support content “suggest method of staying comfortable during congestion to user” instead of the support content “suggest measure to avoid congestion to user.”

When a prediction result of a context is “congestion occurs,” and valence of an estimation result of an affect is low, for example, the setting unit 61 sets support contents “transmit message related to details of prediction result of context as future outlook to user” and “suggest method of staying comfortable during congestion to user.” The selection of the support content may be enabled to be made by referring to the support result table.

For example, when an estimation result of an affect of support corresponding to a support content “transmit message related to details of prediction result of context as future outlook to user” in the support result table is “valence is to drop,” the setting unit 61 sets the support content “suggest method of staying comfortable during congestion to user.” On the other hand, when an estimation result of an affect of support corresponding to the support content “suggest method of staying comfortable during congestion to user” is “valence is to drop,” the setting unit 61 sets the support content “transmit message related to details of prediction result of context as future outlook to user.” According to the above, during a congestion, a future outlook is not transmitted to a user who will feel discomfort when being informed of details of the future outlook but a future outlook is transmitted to a user who will feel at ease by being informed of details of the future outlook. Note that the setting unit 61 may set the support content to “change BGM,” “transmit latest news to user,” and the like.

As described above, the driving support apparatus 303 supports the user based on an estimation result of at least one of an affect and a context and a prediction result of at least one of the affect and the context. Therefore, the user can be supported by predicting a future state of the user. In addition, the driving support apparatus 303 determines a support method and support means by referring to a support result table. Therefore, support with respect to the user can be optimized to the individual.

### Fifth Embodiment

### Configuration Example of Cooking Support System

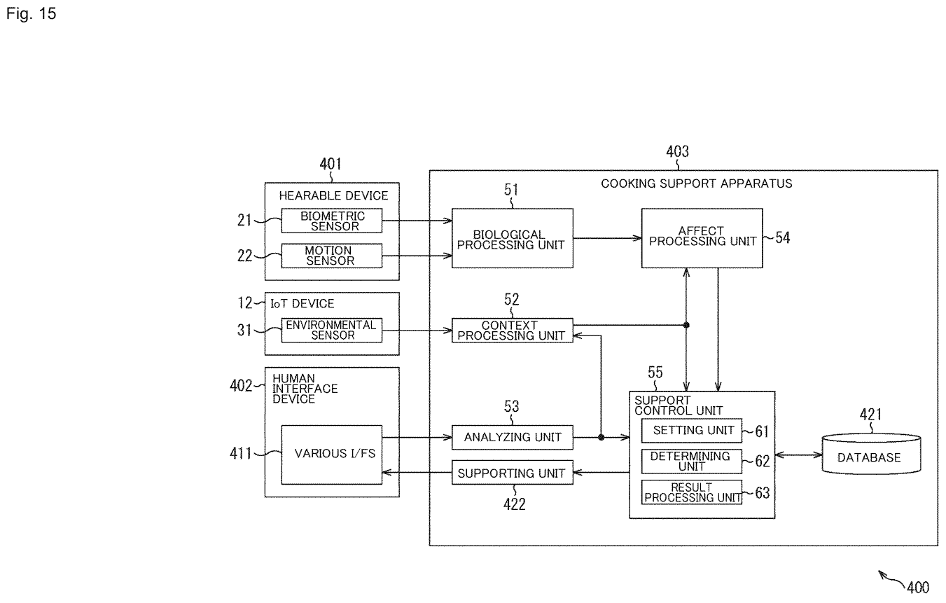

FIG. 15 is a block diagram illustrating a configuration example of a cooking support system including a cooking support apparatus as an information processing apparatus to which the present technique is applied.

In a cooking support system 400 illustrated in FIG. 15, portions corresponding to those of the audio support system 10 illustrated in FIG. 1 are denoted by the same reference signs. Therefore, descriptions of such portions will be omitted as appropriate and the description will focus on the portions that differ from the audio support system 10.

The cooking support system 400 illustrated in FIG. 15 is constituted of one or more hearable devices 401, one or more IoT devices 12, one or more human interface devices 402, and a cooking support apparatus 403. The cooking support system 400 supports the user who is cooking so that the user can cook in a relaxed manner.

Specifically, the hearable device 401 is mounted to an ear of the user. The hearable device 401 includes a biometric sensor 21 and a motion sensor 22 in a similar manner to the wearable device 11. A biosignal acquired by the biometric sensor 21 and associated biological information acquired by the motion sensor 22 are input to the cooking support apparatus 403.

The human interface device 402 includes an input device that accepts an input from the user and an output device which provides the user with output. Examples of the input device include a microphone, a touch sensor, a pressure sensor, and a keyboard and examples of the output device include a speaker, a vibratory apparatus, and a display. The output device may be provided in a smartphone or the like.

The human interface device 402 includes various I/Fs 411 that interact with the user. The various I/Fs 411 of the microphone, the touch sensor, the pressure sensor, and the keyboard input input information to the cooking support apparatus 403 in a similar manner to the various I/Fs 41. The various I/Fs 411 of the speaker, the vibratory apparatus, and the display provides support by outputting audio, vibration, and video with respect to the user based on control information input from the cooking support apparatus 403.

Information exchange between the hearable device 401, the IoT device 12, and the human interface device 402, and the cooking support apparatus 403, is performed via a wired or wireless network.

The cooking support apparatus 403 differs from the audio agent apparatus 14 in that the cooking support apparatus 403 is provided with a database 421 and a supporting unit 422 in place of the database 56 and the supporting unit 57 but is otherwise configured in a similar manner to the audio agent apparatus 14.

While the database 421 of the cooking support apparatus 403 stores a support method table, a support means table, and a support result table in a similar manner to the database 56, the support method table and the support means table are suitable for support by the cooking support apparatus 403 and the support result table corresponds to support by the cooking support apparatus 403.

The supporting unit 422 supports the user by audio, vibration, or video according to the support content supplied from the setting unit 61 and the support method and the support means supplied from the determining unit 62 in a similar manner to the supporting unit 332 illustrated in FIG. 12.

Note that instead of providing the database 421 inside the cooking support apparatus 403, the database 421 may be provided outside the cooking support apparatus 403 and connected to the support control unit 55 via a wired or wireless network.

### Example of Database

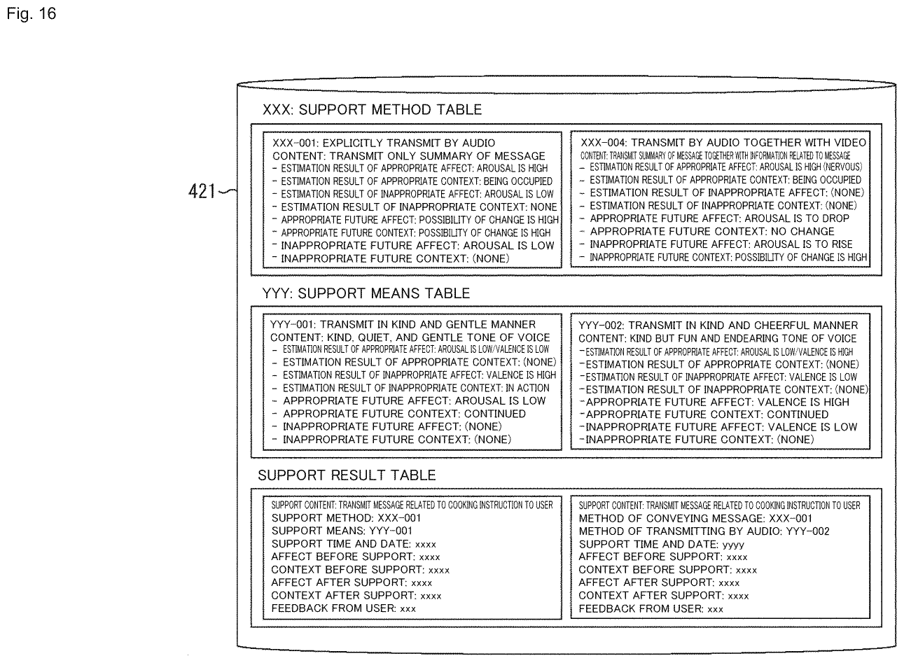

FIG. 16 is a diagram illustrating an example of information stored in the database 421 illustrated in FIG. 15.

FIG. 16 illustrates a support method table corresponding to a support content “transmit message related to cooking instruction to user,” a support means table corresponding to a support method represented by information including “transmit by audio,” and a support result table which are stored in the database 421.

Specifically, in the example illustrated in FIG. 16, in the support method table corresponding to the support content “transmit message related to cooking instruction to user,” a support method with the number “XXX-001” in FIG. 3 is registered.

In association with “transmit by audio together with video” representing a support method, “arousal is high (nervous)” representing an estimation result of an appropriate affect, “being occupied” representing an estimation result of an appropriate context, “(none)” representing an estimation result of an inappropriate affect, “(none)” representing an estimation result of an inappropriate context, “arousal is to drop” representing an appropriate future affect, “no change” representing an appropriate future context, “arousal is to rise” representing an inappropriate future affect, and “possibility of change is high” representing an inappropriate future context are registered. Note that the support method represented by “transmit by audio together with video” is a transmission method of transmitting a message by both video and audio. A number “XXX-004” is attached to this support method.

Support means with the numbers “YYY-001” and “YYY-002” in FIG. 3 are registered in the support means table corresponding to the support method represented by information including “transmit by audio.”

In the support result table, registered in association with the support content “transmit message related to cooking instruction to user,” “XXX-001” representing a support method, and “YYY-001” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Registered in association with the support content “transmit message related to cooking instruction to user,” “XXX-001” representing a support method, and “YYY-002” representing support means are a support time and date at which support embodying the support content had been performed by the support method and the support means, an estimation result of an affect before the support, an estimation result of a context before the support, an estimation result of an affect after the support, an estimation result of a context after the support, and a feedback from the user.

Note that the support method table, the support means table, and the support result table stored in the database 421 are not limited to the example illustrated in FIG. 16.

### Example of Outline of Processing by Cooking Support System

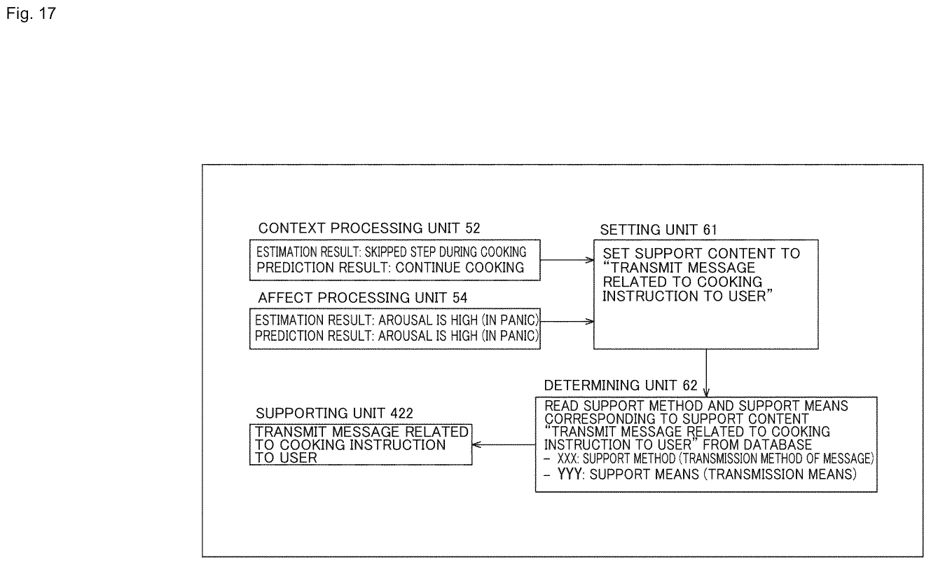

FIG. 17 is a diagram illustrating an example of an outline of processing by the cooking support system 400 illustrated in FIG. 15.

As illustrated in FIG. 17, for example, when an estimation result of a context by the context processing unit 52 is “skipped step during cooking,” a prediction result of a context with high reliability is “continue cooking,” and an estimation result of an affect by the affect processing unit 54 and a prediction result with a high reliability is “arousal is high (in panic),” the setting unit 61 sets the support content to “transmit message related to cooking instruction to user.”

The determining unit 62 selects and reads one support method among support methods corresponding to at least one of an estimation result of an affect and an estimation result of a context from the support method table corresponding to the support content “transmit message related to cooking instruction to user” stored in the database 421. At this point, based on the support result tables, the determining unit 62 selects a support method of which a support result registered in association with the support method is most desirable among the support methods that are selection candidates.

For example, when arousal of the estimation result of an affect is high, the determining unit 62 adopts the support method represented by “clearly transmit by audio” with the number “XXX-001” which is associated with “arousal is high” representing an estimation result of an appropriate affect from the support method table illustrated in FIG. 16 as a selection candidate. In addition, when an estimation result of an affect or a context after support corresponding to the number “XXX-001” in the support result table illustrated in FIG. 16 is an appropriate future affect or context corresponding to the number “XXX-001” in the support method table, the determining unit 62 determines the selection candidate as a current support method and reads the selection candidate.

In addition, the determining unit 62 selects and reads one support means among support means corresponding to at least one of an estimation result of an affect and an estimation result of a context from the support means table corresponding to the current support method which is stored in the database 421. At this point, based on the support result table, the determining unit 62 selects support means of which a support result registered in association with the support means is most desirable among the support means that are selection candidates.

For example, when valence of the estimation result of an affect is low, the determining unit 62 adopts support means “kind, quiet, and gentle tone of voice” with the number “YYY-001” which is associated with “valence is low” representing an estimation result of an appropriate affect as a selection candidate from the support means table illustrated in FIG. 16. In addition, when the estimation result of an affect or a context after support corresponding to the number “YYY-001” in the support result table illustrated in FIG. 16 is an appropriate future affect or context corresponding to the number “YYY-001” in the support means table, the determining unit 62 determines the selection candidate as current support means and reads the selection candidate. Since information representing an appropriate future affect corresponding to the support means with the number “YYY-001” is “arousal is low,” when the support means is determined as the current support means, the cooking support apparatus 403 can support the panicking user so as to calm the user down.

Based on the support content set by the setting unit 61 and the support method and the support means determined by the determining unit 62, the supporting unit 422 performs support of transmitting a message related to a cooking instruction to the user as cooking support information for supporting cooking by the user. Specifically, the supporting unit 422 generates control information based on the support content, the support method, and the support means and supplies the control information to the various I/Fs 411.

For example, when the support method represented by “explicitly transmit by audio” is determined as the current support method and the support means “kind, quiet, and gentle tone of voice” is determined as the current support means, the supporting unit 422 generates control information for causing the speaker to output only a summary of a message related to a cooking instruction in a kind, quiet, and gentle tone of voice and supplies the control information to the various I/Fs 411 of the speaker. Accordingly, audio that transmits only a summary of a message related to a cooking instruction in a kind, quiet, and gentle tone of voice is output from the speaker. Note that audio that transmits only a summary of a message related to a cooking instruction is, for example, audio that explicitly transmits a cooking instruction “Let's do step A.”

On the other hand, audio that transmits a message related to a cooking instruction to the user according to the support method represented by “transmit by audio together with video” is, for example, audio that explicitly transmits a cooking instruction and prompts the user to look at the display such as “Let's do step A. Look at the display.” When performing the support according to the support method, the supporting unit 422 also generates control information for controlling the display so as to display a video representing the cooking instruction and supplies the control information to the various I/Fs 411 of the display. As a result, audio of “Let's do step A. Look at the display” is output from the speaker and, at the same time, a video representing the cooking instruction is displayed on the display. In other words, support of transmitting a message related to the cooking instruction to the user is performed by audio and video.

Note that the cooking instruction to be transmitted to the user is, for example, a popular cooking instruction for a dish that the user wants to make which has been acquired via the Internet or the like.

After support by the supporting unit 422, the result processing unit 63 generates a support result based on an estimation result of at least one of an affect and a context of the user before and after the support and an analysis result of input information supplied from the analyzing unit 53. In addition, the result processing unit 63 registers the support result in the support result table in the database 421 in association with the current support content, the current support method, and the current support means. For example, when the current support method is a support method represented by “explicitly transmit by audio” and the current support means is the support means “kind, quiet, and gentle tone of voice,” a leftmost piece of information in the support result table illustrated in FIG. 16 is registered.

The support result table is to be used when determining a support method and support means of a next support. For example, when arousal of an estimation result of an affect after the current support is high or, in other words, when the user is feeling anxious, the determining unit 62 determines support means of which arousal of an estimation result of an affect after the support is low in the support result table such as support means “gentle tone of voice” as next support means. When arousal is high even after support is performed by the support means “gentle tone of voice,” the determining unit 62 determines another support method of which arousal of an estimation result of an affect after the support is low in the support result table such as the support method of “XXX-003” in FIG. 16 as a next support method. In this manner, a cooking instruction can be transmitted using also video to a user who feels anxious when the cooking instruction is transmitted by audio.

When an estimation result of a context is “skipped step during cooking” or “performed wrong step,” a prediction result is “continue cooking,” and an estimation result of an affect and arousal of a prediction result are low, for example, while the support content and the support method are the same as when arousal is high, support means corresponding to “arousal is high” representing an appropriate future affect such as support means “firm tone of voice” is determined as the support means. Accordingly, support is performed with respect to a user having skipped a step during cooking or performed a wrong step due to arousal being low or, in other words, a lack of concentration so as to restore the concentration.

When arousal of an estimation result of an affect after support corresponding to the support content, the support method, and the support means rises as compared to before the support, the support is determined to be effective. In addition, by referring to the support result table of the support, during support when at least one of an estimation result and a prediction result of a context and an estimation result and a prediction result of an affect is the same as the current support, support of which the support content, the support method, and the support means are the same as the current support is performed.

On the other hand, when arousal of an estimation result of an affect after support does not rise as compared to before the support, for example, the setting unit 61 sets another support content corresponding to “arousal is high” representing an estimation result of the affect after support in the support result table as a next support content. Examples of the next support content include support contents “output BGM with aggressive rhythm,” “suggest that user do some stretches,” “suggest that user have something to drink,” and “suggest that user take short break.” As described above, support to raise arousal of the user or, in other words, support to restore concentration is performed by referring to the support result table and setting a support content with the setting unit 61.

As described above, the cooking support apparatus 403 supports the user based on an estimation result of at least one of an affect and a context and a prediction result of at least one of the affect and the context. Therefore, the user can be supported by predicting a future state of the user. In addition, the cooking support apparatus 403 determines a support method and support means by referring to a support result table. Therefore, support with respect to the user can be optimized to the individual.

While the affect processing unit 54 estimates a present affect of the user based on a feature amount of a biosignal in the description given above, the present affect of the user can also be directly estimated from a biosignal using a DNN (Deep Neural Network) or the like. In this case, the biological processing unit 51 only performs preprocessing such as noise removal and resampling based on associated biological information and does not perform processing of extracting a feature amount from the biosignal. In addition, the affect processing unit 54 may be configured to obtain an estimation result and a prediction result of an affect using a model other than Russell's circumplex model of affect.

### Description of Computer

The above-described series of processing of the audio agent apparatus 14, the navigation apparatus 104, the pet-type robot agent apparatus 212, the driving support apparatus 303, and the cooking support apparatus 403 can also be performed by hardware or software. In a case in which the series of processing is performed by software, a program constituting the software is installed in a computer. In this case, the computer includes a computer embedded in dedicated hardware or, for example, a general-purpose computer capable of executing various functions by installing various programs.

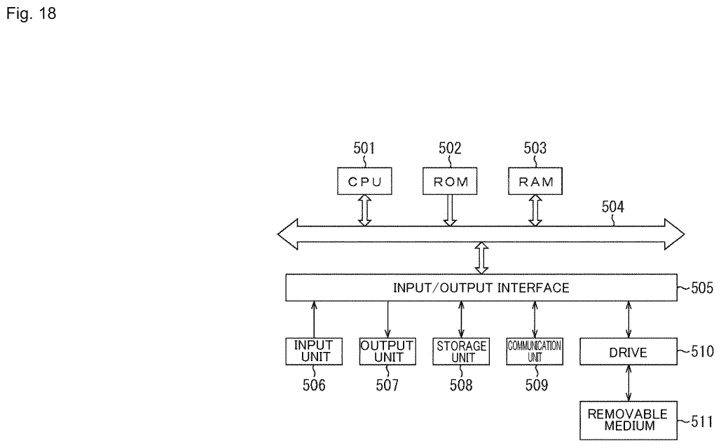

FIG. 18 is a block diagram illustrating a hardware configuration example of a computer that executes the series of processing of the audio agent apparatus 14, the navigation apparatus 104, the pet-type robot agent apparatus 212, the driving support apparatus 303, and the cooking support apparatus 403 described above according to a program.

In the computer, a CPU (Central Processing Unit) 501, a ROM (Read Only Memory) 502, and a RAM (Random Access Memory) 503 are connected to one another by a bus 504.

An input/output interface 505 is further connected to the bus 504. An input unit 506, an output unit 507, a storage unit 508, a communication unit 509, and a drive 510 are connected to the input/output interface 505.

The input unit 506 is a keyboard, a mouse, a microphone, or the like. The output unit 507 is a display, a speaker, or the like. The storage unit 508 is a hard disk, non-volatile memory, or the like. The communication unit 509 is a network interface or the like. The drive 510 drives a removable medium 511 such as a magnetic disk, an optical disc, a magneto-optical disk, or a semiconductor memory.

In the computer configured as described above, for example, the CPU 501 performs the above-described series of processing by loading a program stored in the storage unit 508 onto the RAM 503 via the input/output interface 505 and the bus 504 and executing the program.

The program executed by the computer (the CPU 501) can be recorded on, for example, the removable medium 511 serving as a package medium for supply. In addition, the program can be supplied via a wired or wireless transfer medium such as a local area network, the Internet, or digital satellite broadcasting.

In the computer, by mounting the removable medium 511 on the drive 510, it is possible to install the program in the storage unit 508 via the input/output interface 505. The program can be received by the communication unit 509 via a wired or wireless transfer medium to be installed in the storage unit 508. In addition, the program may be installed in advance in the ROM 502 or the storage unit 508.

Note that the program executed by a computer may be a program that performs processing chronologically in the order described in the present specification or may be a program that performs processing in parallel or at a necessary timing such as a called time.

The present technique can be applied to a user-supporting agent system that supports a user other than the audio support system, the bicycle navigation system, the pet-type robot system, the driving support system, and the cooking support system described above. For example, the present technique can be applied to other task support systems which support studying and working as tasks performed by the user instead of driving and cooking. Such task support systems are to be configured in a similar manner to the cooking support system 400 with the exception of studying and working replacing cooking as the task. Since the driving support information and the cooking support information described above are both support information that supports a task by the user, the pieces of information can be collectively referred to as task support information that supports a task by the user. For example, a projector or the like may be used as the output device.

In the present specification, a system refers to a collection of a plurality of constituent elements (apparatuses, modules (components), or the like) and all the constituent elements may or may not be located in a same casing. Accordingly, a plurality of apparatuses stored in separate casings and connected via a network and a single apparatus in which a plurality of modules are stored in one casing are all systems.

Embodiments of the present technique are not limited to the aforementioned embodiments and various changes can be made without departing from the gist of the present technique.

For example, a combination of all or part of the above-mentioned plurality of embodiments may be employed.

For example, the present technique may be configured as cloud computing in which a plurality of apparatuses share and cooperatively process one function via a network.

In addition, each step described in the above flowchart can be executed by one apparatus or executed in a shared manner by a plurality of apparatuses.

Furthermore, in a case in which one step includes a plurality of processes, the plurality of processes included in the one step can be executed by one apparatus or executed in a shared manner by a plurality of apparatuses.

The advantageous effects described in the present specification are merely exemplary and are not limited, and other advantageous effects of the advantageous effects described in the present specification may be achieved.

The present technique can be configured as follows.
(1)

An information processing apparatus, including:
a supporting unit configured to support a user based on an estimation result of at least one of a present affect and a present context of the user and a prediction result of at least one of a future affect and a future context of the user.
(2)

The information processing apparatus according to (1), further including:
an affect processing unit configured to obtain the prediction result of the affect based on the estimation result of the affect and at least one of the estimation result and the prediction result of the context.
(3)

The information processing apparatus according to (2), wherein
the affect processing unit is further configured to obtain a reliability of the prediction result of the affect based on the estimation result of the affect and at least one of the estimation result and the prediction result of the context.
(4)

The information processing apparatus according to (1), further including:
an affect processing unit configured to obtain the estimation result of the affect using a biosignal of the user.
(5)

The information processing apparatus according to any one of (1) to (4), further including:
a context processing unit configured to obtain the prediction result of the context based on estimation results of the context in a time series.
(6)

The information processing apparatus according to any one of (1) to (4), further including:
a context processing unit configured to obtain the estimation result of the context based on at least one of environmental information indicating an environment surrounding the user and input information that is information input from the user.
(7)

The information processing apparatus according to any one of (1) to (6), further including:
a setting unit configured to set a support content that is a content of support by the supporting unit based on the estimation result of at least one of the affect and the context and the prediction result of at least one of the affect and the context,
wherein
the supporting unit is configured to perform support of the support content set by the setting unit.
(8)

The information processing apparatus according to (7), further including:
a determining unit configured to determine a support method that is a method of support by the supporting unit based on the estimation result of at least one of the affect and the context, wherein
the supporting unit is configured to perform support of the support content by the support method determined by the determining unit.
(9)

The information processing apparatus according to (8), further including:
a result processing unit configured to cause a storage unit to store a support result with respect to the user in association with the support method based on an estimation result of at least one of the affect and the context before and after the support by the supporting unit.
(10)

The information processing apparatus according to (9), wherein
the determining unit is configured to determine the support method also based on the support result stored in the storage unit.
(11)

The information processing apparatus according to (8), wherein
the determining unit is configured to also determine support means that is means of support by the supporting unit based on an estimation result of at least one of the affect and the context, and
the supporting unit is configured to perform support of the support content by the support method and the support means determined by the determining unit.
(12)

The information processing apparatus according to (11), further including
a result processing unit configured to cause a storage unit to store a support result with respect to the user in association with the support method and the support means based on an estimation result of at least one of the affect and the context before and after the support by the supporting unit.
(13)

The information processing apparatus according to (12), wherein
the determining unit is configured to determine the support method and the support means also based on the support result stored in the storage unit.
(14)

The information processing apparatus according to any one of (11) to (13), wherein the support content is to transmit a message to the user,
the support method is a method of transmitting the message to the user by audio, and
the support means is a tone of voice or a volume of the audio.
(15)

The information processing apparatus according to (14), wherein
the message is navigation information.
(16)

The information processing apparatus according to (14), wherein
the message is task support information that supports a task by the user.
(17)

The information processing apparatus according to any one of (11) to (13), wherein the support content is to make a suggestion to the user,
the support method is a method of making a suggestion to the user via a motion of a robot, and
the support means is a predetermined motion of the robot.
(18)

An information processing method including an information processing apparatus performing
a supporting step of supporting a user based on an estimation result of at least one of a present affect and a present context of the user and a prediction result of at least one of a future affect and a future context of the user.
(19)

A program causing a computer to function as:
a supporting unit configured to support a user based on an estimation result of at least one of a present affect and a present context of the user and a prediction result of at least one of a future affect and a future context of the user.

### REFERENCE SIGNS LIST

14 Audio agent apparatus
52 Context processing unit
54 Affect processing unit
56 Database
57 Supporting unit
61 Setting unit
62 Determining unit
63 Result processing unit
104 Navigation apparatus
131 Context processing unit
132 Database
133 Supporting unit
212 Pet-type robot agent apparatus
231 Database
232 Supporting unit
303 Driving support apparatus
331 Database
332 Supporting unit
403 Cooking support apparatus
421 Database
422 Supporting unit

1. An information processing apparatus, comprising:
a supporting unit configured to support a user based on an estimation result of at least one of a present affect and a present context of the user and a prediction result of at least one of a future affect and a future context of the user.

2. The information processing apparatus according to claim 1, further comprising:
an affect processing unit configured to obtain the prediction result of the affect based on the estimation result of the affect and at least one of the estimation result and the prediction result of the context.

3. The information processing apparatus according to claim 2, wherein
the affect processing unit is further configured to obtain a reliability of the prediction result of the affect based on the estimation result of the affect and at least one of the estimation result and the prediction result of the context.

4. The information processing apparatus according to claim 1, further comprising:
an affect processing unit configured to obtain the estimation result of the affect using a biosignal of the user.

5. The information processing apparatus according to claim 1, further comprising:
a context processing unit configured to obtain the prediction result of the context based on estimation results of the context in a time series.

6. The information processing apparatus according to claim 1, further comprising:
a context processing unit configured to obtain the estimation result of the context based on at least one of environmental information indicating an environment surrounding the user and input information that is information input from the user.

7. The information processing apparatus according to claim 1, further comprising:
a setting unit configured to set a support content that is a content of support by the supporting unit based on the estimation result of at least one of the affect and the context and the prediction result of at least one of the affect and the context,
wherein
the supporting unit is configured to perform support of the support content set by the setting unit.

8. The information processing apparatus according to claim 7, further comprising:
a determining unit configured to determine a support method that is a method of support by the supporting unit based on the estimation result of at least one of the affect and the context, wherein
the supporting unit is configured to perform support of the support content by the support method determined by the determining unit.

9. The information processing apparatus according to claim 8, further comprising:
a result processing unit configured to cause a storage unit to store a support result with respect to the user in association with the support method based on an estimation result of at least one of the affect and the context before and after the support by the supporting unit.

10. The information processing apparatus according to claim 9, wherein
the determining unit is configured to determine the support method also based on the support result stored in the storage unit.

11. The information processing apparatus according to claim 8, wherein
the determining unit is configured to also determine support means that is means of support by the supporting unit based on an estimation result of at least one of the affect and the context, and
the supporting unit is configured to perform support of the support content by the support method and the support means determined by the determining unit.

12. The information processing apparatus according to claim 11, further comprising
a result processing unit configured to cause a storage unit to store a support result with respect to the user in association with the support method and the support means based on an estimation result of at least one of the affect and the context before and after the support by the supporting unit.

13. The information processing apparatus according to claim 12, wherein
the determining unit is configured to determine the support method and the support means also based on the support result stored in the storage unit.

14. The information processing apparatus according to claim 11, wherein
the support content is to transmit a message to the user,
the support method is a method of transmitting the message to the user by audio, and
the support means is a tone of voice or a volume of the audio.

15. The information processing apparatus according to claim 14, wherein
the message is navigation information.

16. The information processing apparatus according to claim 14, wherein
the message is task support information that supports a task by the user.

17. The information processing apparatus according to claim 11, wherein
the support content is to make a suggestion to the user,
the support method is a method of making a suggestion to the user via a motion of a robot, and
the support means is a predetermined motion of the robot.

18. An information processing method, comprising an information processing apparatus performing
a supporting step of supporting a user based on an estimation result of at least one of a present affect and a present context of the user and a prediction result of at least one of a future affect and a future context of the user.

19. A program causing a computer to function as:
a supporting unit configured to support a user based on an estimation result of at least one of a present affect and a present context of the user and a prediction result of at least one of a future affect and a future context of the user.
